(19) United States
(12) Patent Application Publication
(10) Pub. No.: US 2023/0143949 A1
(43) Pub. Date: May 11, 2023

(54) MICROBEADS AND USES THEREOF

(71) Applicant: Shanghai Polaris Biology Co., Ltd., Shanghai (CN)

(72) Inventors: Ran Wei, Shanghai (CN); Shuangwu Sun, Shanghai (CN); Yuchong Wang, Shanghai (CN); Ziyang Ge, Shanghai (CN)

(21) Appl. No.: 18/055,690

(22) Filed: Nov. 15, 2022

Related U.S. Application Data
(63) Continuation of application No. PCT/CN2021/094398, filed on May 18, 2021.

(30) Foreign Application Priority Data
May 18, 2020 (CN) PCT/CN2020/90905

(51) Int. Cl. G01N 33/543; G01N 33/53; G01N 33/60; G01N 33/68

(52) U.S. Cl. CPC G01N 33/54313; G01N 33/5308; G01N 33/60; G01N 33/6848

(57) ABSTRACT

The present disclosure provides a microbead and a method for preparing the same. Also provided is a method for detecting and analyzing biomolecules in sample by using element analysis.

### CROSS-REFERENCE

This application is a continuation application of International Application No. PCT/CN2021/094398, filed May 18, 2021, which claims priority to International Application No. PCT/CN2020/090905, filed May 18, 2020, the content of which application is incorporated herein by reference.

### BACKGROUND

Mass Cytometry is a mass spectrometry technique based on element analysis, which has been used for determination of cells. In this approach, cellular affinity molecules such as antibodies are conjugated with an element, and these cellular affinity molecules are further used to label a target cell which is further subject to Mass Cytometry and analysis based on the signal of the element. Compared to the conventional Flow Cytometry, this approach utilizes elements or isotopes as the reporter system instead of fluorescence, which provides a broader spectrum while maintaining its high accuracy and sensitivity.

Detection and/or identification of biomolecules is essential in medical fields, including diagnostics, toxicology, and pathology. Development of technologies in detecting and analyzing biomolecules have led to unprecedented advances in understanding the mechanisms of health, disease and treatment. However, deficiencies of these technologies are mostly related to limitations in sensitivity, accuracy, selectivity, as well as the ability to determine the quantities of multianalytes simultaneously. There exists a need of a method for detecting biomolecules in a more accurate, cost effective, and high throughput manner.

### SUMMARY

Recognized herein is a need for a method and composition for detecting multiple biomolecules simultaneously in an accurate, cost effective, and high throughput manner. The present invention addresses this need and provides related advantages as well.

In one aspect, provided is a microbead comprising a plurality of functional groups on surface of a microbead substrate, wherein a first subset of the plurality of the functional groups is attached to a chelating group capable of chelating with an element tag, and a second subset of the plurality of the functional groups is coupled with an affinity moiety capable of binding to a target molecule. In some embodiments, the microbead substrate is formed by a polymer selected from the group consisting of polystyrene (PS), polystyrene-methacrylic acid (PSMAA), polystyrene-divinylbenzene (P[S/DVB]), polymethyl methacrylate (PMMA), and any combination thereof. In some embodiments, the microbead substrate is formed by polystyrene (PS). In some embodiments, the microbead has a diameter between 50 nm-10 μm. In some embodiments, the microbead has a diameter between 1-3 μm. In some embodiments, the functional group is selected from the group consisting of carboxyl, sulfhydryl, amino, hydroxyl, maleimide, azide, alkynyl, biotin and any combination thereof. In some embodiments, the functional group is carboxyl.

In some embodiments, the microbead comprises 10⁴-10¹⁰ functional groups on its surface. In some embodiments, the microbead comprises 10⁶-10⁹ functional groups on its surface. In some embodiments, the microbead comprises 10⁴-10¹⁰ chelating groups attached to the functional groups. In some embodiments, the microbead comprises 10⁶-10⁹ chelating groups attached to the functional groups. In some embodiments, the microbead comprises 10⁴-10¹⁰ affinity moieties coupled with the functional groups. In some embodiments, the microbead comprises 10⁵-10⁶ affinity moieties coupled with the functional groups.

In some embodiments, the affinity moiety is selected from the group consisting of peptide, protein, aptamer, antibody, enzyme, avidin, streptavidin, carbohydrate, nucleic acid, deoxyribonucleic acid, oligonucleotide, polypeptide, recombinant protein, ribonucleic acid lipid, and a derivative thereof. In some embodiments, the antibody is selected from a group consisting of monoclonal antibody, polyclonal antibody, antibody fragment, Fab fragment, Fc fragment, light chain, heavy chain, immunoglobin, and immunoglobin fragment. In some embodiments, the affinity moiety is directly coupled with the functional group. In some embodiments, the affinity moiety is coupled with the functional group via a spacer. In some embodiments, the spacer comprises at least 2 different spacers. In some embodiments, the spacer selected from MPBH (4-(4-N-maleimidophenyl) butyric acid hydrazide) or NH₂-(PEG)ₙ-Maleimide. In some embodiments, he chelating group is selected from the group consisting of EDTA, DTPA, DCTA, DOTA, TETA, NOTA, and a derivative thereof. In some embodiments, the chelating group is directly attached to the functional group. In some embodiments, chelating group is attached to the functional group via a spacer. In some embodiments, the spacer comprises at least 2 different spacers. In some embodiments, the spacer is selected from MPBH (4-(4-N-maleimidophenyl) butyric acid hydrazide) or NH₂-(PEG)ₙ-Maleimide.

In some embodiments, the chelating group of the microbead further chelates with an element tag. In some embodiments, the element tag comprises an element or a combination of elements. In some embodiments, the combination of elements is a combination of two or more elements. In some embodiments, the combination of elements is a combination of three or more elements. In some embodiments, the element is a metal or an isotope thereof. In some embodiments, the metal is selected from the group consisting of Y, Ru, Rh, Pd, Cd, In, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og. In some embodiments, the isotope is selected from the group consisting of Y-89, Rh103, Pd-102, Pd-104, Pd-105, Pd106, Pd-108, Pd-110, Cd-106, Cd-108, Cd-110, Cd-111, Cd-112, Cd-113, Cd-114, Cd-116, In-115, La-139, Ce-140, Ce-142, Pr-141, Nd-142, Nd-143, Nd-144, Nd-145, Nd-146, Nd-148, Nd-150, Sm-144, Sm-147, Sm-148, Sm-149, Sm-150, Sm-152, Sm-154, Eu-151, Eu-153, Gd-154, Gd-155, Gd-156, Gd-157, Gd-158, Gd-160, Tb-159, Dy-160, Dy-161, Dy-162, Dy-163, Dy-164, Ho-165, Er-164, Er-166, Er-167, Er-168, Er-170, Tm-169, Yb-170, Yb-171, Yb-172, Yb-173, Yb-174, Yb-176, Lu-175, Lu-176 and Bi-209.

In another aspect, provided, is a method for preparing a combinational labeling microbead, comprising: (a) providing a polymeric microbead substrate; (b) functionalizing the polymeric microbead substrate to provide a plurality of functional groups on the surface of the polymeric microbead substrate; (c) reacting a chelating group with the polymeric microbead substrate in (b), so that the chelating group is attached to a first subset of the functional groups on surface of the polymeric microbead substrate; (d) reacting an affinity moiety with the polymeric microbead substrate in (b), so that the affinity moiety is coupled with a second subset of the functional groups on surface of the polymeric microbead substrate. In some embodiments, the method further comprises chelating an element tag to the chelating group. In some embodiments, the element tag comprises an element or a combination of elements. In some embodiments, the combination of elements is a combination of two or more elements. In some embodiments, the combination of elements is a combination of three or more elements. In some embodiments, the element is a metal or an isotope thereof. In some embodiments, the polymeric microbead substrate is provided by polymerization of styrene, styrene-methacrylic acid (SMAA), styrene-divinylbenzene (S/DVB), and methyl methacrylate (PMMA).

In some embodiments, 10⁴-10¹⁰ of the functional groups are provided on the surface of the polymeric microbead substrate. In some embodiments, 10⁶-10⁹ of the functional groups are provided on the surface of the polymeric microbead substrate. In some embodiments, the functional group provided on the surface of the polymeric microbead is selected from the group consisting of carboxyl, sulfhydryl, amino, hydroxyl, maleimide, azide, alkynyl, biotin, avidin, streptavidin, and any combination thereof. In some embodiments, the functional group provided on the surface of the polymeric microbead is carboxyl.

In some embodiments, the chelating group is attached to the functional group via a spacer. In some embodiments, n the spacer is selected from MPBH (4-(4-N-maleimidophenyl) butyric acid hydrazide) or NH₂-(PEG)ₙ-Maleimide. In some embodiments, the chelating group is attached to the functional group via an amine of the chelating group. In some embodiments, the amine is a primary amine. In some embodiments, the chelating group is attached to the functional group through carbodiimide crosslinking. In some embodiments, the carbodiimide crosslinking comprises activating the functional group by a carbodiimide compound before attaching the chelating group to the functional group. In some embodiments, the carbodiimide compound is selected from EDC (1-Ethyl-3-[3-dimethylaminopropyl] carbodiimide hydrochloride) and DCC (dicyclohexyl carbodiimide). In some embodiments, the carbodiimide crosslinking further comprises forming an NETS-ester intermediate before attaching the chelating group to the functional group. In some embodiments, the NETS-ester intermediate is formed by NETS (N-hydroxy-succinimide) or Sulfo-NETS (N-hydroxy-sulfo-succinimide).

In some embodiments, the affinity moiety is coupled with the functional group via a spacer. In some embodiments, the spacer is selected from MPBH (4-(4-N-maleimidophenyl) butyric acid hydrazide) or NH₂-(PEG)ₙ-Maleimide. In some embodiments, the affinity moiety is coupled with the functional group via an amine of the affinity molecule. In some embodiments, the amine is a primary amine. In some embodiments, the affinity moiety is coupled with the functional group through carbodiimide crosslinking. In some embodiments, the carbodiimide crosslinking comprises activating the functional group by a carbodiimide compound before coupling the affinity moiety with the functional group. In some embodiments, the carbodiimide compound is selected from EDC (1-Ethyl-3-[3-dimethylaminopropyl] carbodiimide hydrochloride) and DCC (dicyclohexyl carbodiimide). In some embodiments, the carbodiimide crosslinking further comprises forming an NETS-ester intermediate before coupling the affinity moiety with the functional group. In some embodiments, the NETS-ester intermediate is formed by NETS (N-hydroxy-succinimide) or Sulfo-NETS (N-hydroxy-sulfo-succinimide). In some embodiments, the affinity moiety is coupled with the functional group before attaching the chelating group to the functional group. In some embodiments, the affinity moiety is coupled with the functional group after attaching the chelating group to the functional group. In some embodiments, the affinity moiety is coupled with the functional group before chelating the element tag to the chelating group. In some embodiments, the affinity moiety is coupled with the functional group after chelating the element tag to the chelating group.

In another aspect, provided is a population of microbeads comprising two or more subpopulations of the microbeads, wherein at least one of the microbeads comprises a plurality of functional groups on surface of a microbead substrate, a first subset of the plurality of the functional groups is attached to a chelating group chelated with an element tag, and a second subset of the plurality of the functional groups is coupled with an affinity moiety capable of binding to a target molecule; and wherein at least one subpopulation of the microbeads comprises an element tag that is different from at least another subpopulation of the microbeads, and at least one subpopulation of the microbeads comprises an affinity moiety that is different from another subpopulation of the microbeads.

In some embodiments, the element tag is a combination of elements. In some embodiments, the combination of elements is a combination of two or more elements. In some embodiments, the combination of elements is a combination of three or more elements. In some embodiments, the element is a metal or an isotope thereof. In some embodiments, the element tag of each subpopulation of the microbeads functions as a barcode for the affinity moiety of the microbeads in an element analysis.

In some embodiments, the element analysis is MS. In some embodiments, the MS is selected from the group consisting of ICP-MS, ICP-TOF-MS and Mass Cytometry. In some embodiments, the population of the microbeads has a variable coefficient of diameter (CVd) that is less than 10%. In some embodiments, the population of the microbeads has a variable coefficient of diameter (CVd) that is less than 5%.

In another aspect, provided is a method for detecting an analyte in a sample, comprising: (a) contacting the sample with a microbead, wherein the microbead comprises a plurality of functional groups on surface of a microbead substrate, and a first subset of the plurality of the functional groups is attached to a chelating group chelated with an element tag and a second subset of the plurality of the functional groups is coupled with an affinity moiety capable of binding to the analyte; (b) differentiating the microbead bound to the analyte from the microbead not bound to the analyte; (c) determining the analyte in the sample.

In some embodiments, the differentiating comprises incubating the microbead with an immobilizing reagent, wherein the immobilizing reagent specifically binds to the analyte and immobilizes the microbead bound to the analyte. In some embodiments, the method further comprises isolating the microbead bound to the analyte. In some embodiments, the differentiating comprises incubating the microbead with a detecting reagent, wherein the detecting reagent specifically binds to the analyte and allows for detection of the microbead bound to the analyte. In some embodiments, the method further comprises isolating the microbead from the sample. In some embodiments, the detecting reagent is selected from the group consisting of peptide, protein, aptamer, antibody, enzyme, carbohydrate, nucleic acid, deoxyribonucleic acid, oligonucleotide, polypeptide, recombinant protein, ribonucleic acid lipid, and a derivative thereof. In some embodiments, the detecting reagent is an antibody.

In some embodiments, the detecting reagent is labeled with a detecting element tag. In some embodiments, the detecting element tag is a metal or an isotope thereof. In some embodiments, the metal is selected from the group consisting of Y, Ru, Rh, Pd, Cd, In, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og. In some embodiments, the isotope is selected from the group consisting of Y-89, Rh103, Pd-102, Pd-104, Pd-105, Pd106, Pd-108, Pd-110, Cd-106, Cd-108, Cd-110, Cd-111, Cd-112, Cd-113, Cd-114, Cd-116, In-115, La-139, Ce-140, Ce-142, Pr-141, Nd-142, Nd-143, Nd-144, Nd-145, Nd-146, Nd-148, Nd-150, Sm-144, Sm-147, Sm-148, Sm-149, Sm-150, Sm-152, Sm-154, Eu-151, Eu-153, Gd-154, Gd-155, Gd-156, Gd-157, Gd-158, Gd-160, Tb-159, Dy-160, Dy-161, Dy-162, Dy-163, Dy-164, Ho-165, Er-164, Er-166, Er-167, Er-168, Er-170, Tm-169, Yb-170, Yb-171, Yb-172, Yb-173, Yb-174, Yb-176, Lu-175, Lu-176 and Bi-209.

In some embodiments, the determining the analyte comprises determining an identity of the analyte in the sample. In some embodiments, the determining the identity of the analyte is performed by analyzing the identity of the element tag. In some embodiments, the determining the analyte comprises quantifying the amount of the analyte in the sample. In some embodiments, the quantifying the amount of the analyte is performed by analyzing the detecting element tag. In some embodiments, the determining the analyte is performed by an element analysis. In some embodiments, the element analysis is MS. In some embodiments, the MS is selected from the group consisting of ICP-MS, ICP-TOF-MS and Mass Cytometry.

In some embodiments, the sample is obtained from a subject. In some embodiments, the sample is bodily fluid. In some embodiments, the bodily fluid is selected from the group consisting of whole blood, plasma, serum, urine, effusions, ascitic fluid, saliva, cerebrospinal fluid, cervical secretions, vaginal secretions, endometrial secretions, amniotic fluid, gastrointestinal secretions, bronchial secretions including sputum, breast fluid and secretions. In some embodiments, the analyte is one or more biomarkers for predicting or diagnosing a disease. In some embodiments, biomarker is selected from the group consisting of cytokines, chemokines, growth factors, proteins, peptides, nucleic acids, oligonucleotides, and metabolites.

In another aspect, provided is a method of performing a multiplex analysis of two or more analytes in a sample, comprising: (a) contacting a population of microbeads with the sample, wherein at least one of the microbeads comprises a plurality of functional groups on surface of the microbead substrate; a first subset of the plurality of the functional groups is attached to a chelating group chelated with an element tag; a second subset of the plurality of the functional groups is coupled with an affinity moiety capable of binding to an analyte of the two or more analytes, wherein at least one subpopulation of the microbeads comprises the element tag that is different from at least another subpopulation of the microbeads, and at least one subpopulation of the microbeads comprises the affinity moiety that is different from another subpopulation of the microbeads; (b) incubating the population of the microbeads with two or more detecting reagents, wherein each of the detecting reagents allows for detection of a microbead bound to an analyte; (c) simultaneously analyzing the two or more analytes in the sample. In some embodiments, he element tag is a combination of elements. In some embodiments, the combination of elements is a combination of two or more elements. In some embodiments, the combination of elements is a combination of three or more elements. In some embodiments, the method further comprises isolating the microbead from the sample. In some embodiments, the detecting reagent is labeled with a detecting element tag.

In some embodiments, the analyzing the two or more analytes comprises determining the identities of the two or more analytes in the sample. In some embodiments, the determining the identities of the two or more analytes is performed by analyzing the identity of the element tag. In some embodiments, the analyzing the two or more analytes comprises quantifying the amounts of the two or more analytes in the sample. In some embodiments, the quantifying the amounts of the two or more analytes is performed by analyzing the detecting element tag. In some embodiments, the analyzing the two or more analytes is performed by an element analysis. In some embodiments, the element analysis is MS. In some embodiments, the MS is selected from the group consisting of ICP-MS, ICP-TOF-MS and Mass Cytometry.

In some embodiments, the sample is obtained from a subject. In some embodiments, the sample is bodily fluid. In some embodiments, the bodily fluid is selected from the group consisting of whole blood, plasma, serum, urine, effusions, ascitic fluid, saliva, cerebrospinal fluid, cervical secretions, vaginal secretions, endometrial secretions, amniotic fluid, gastrointestinal secretions, bronchial secretions including sputum, breast fluid and secretions. In some embodiments, each of the two or more analytes is a biomarker for predicting or diagnosing a disease. In some embodiments, the biomarker is selected from the group consisting of cytokines, chemokines, growth factors, proteins, peptides, nucleic acids, oligonucleotides, and metabolites.

In another aspect, provided is a kit comprising: (1) an instruction for performing a multiplex analysis of two or more analytes by using the kit, (2) a population of microbeads comprising two or more subpopulations of the microbeads, wherein at least one of the microbeads comprises a plurality of functional groups on surface of a microbead substrate, a first subset of the plurality of the functional groups are attached to a chelating group chelated with an element tag, and a second subset of the plurality of the functional groups is coupled with an affinity moiety capable of binding to a target molecule, and wherein at least one subpopulation of the microbeads comprises an element tag that is different from at least another subpopulation of the microbeads, and at least one subpopulation of the microbeads comprises an affinity moiety that is different from another subpopulation of the microbeads, and (3) two or more detecting reagents, wherein each of the detecting reagents comprises a detecting element tag and specifically binds to an analyte of the two or more analytes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic diagram of the microbead of the present disclosure.

FIG. 2A illustrates exemplary processes for coupling the affinity moiety with the functional groups on the surface of the microbead. FIG. 2B illustrates exemplary processes for attaching the chelating groups to the functional groups on the surface of the microbead. FIG. 2C illustrates exemplary spacers that can be used to conjugate the affinity moiety to the functional group on the surface of the microbead.

FIG. 3 illustrates analysis of the microbead with a combinational element tag (141Pr, 159Tb and 166Ho) by using ICP-TOF-MS.

FIG. 4 illustrates analysis of the microbead with a combinational element tag (141Pr, 159Tb and 166Ho) and mouse IgG (labeled with 169Tm) by using Mass Cytometry.

FIG. 5A illustrates coupling efficiency of the microbeads with mouse IgG at different concentrations. FIG. 5B illustrates quantification of the detecting element tag in a linear coordinate. FIG. 5C illustrates quantification of the detecting element tag in a logarithmic coordinate.

FIG. 6A illustrates detection of human TNFα in the sample by using the microbead through Mass Cytometry. FIG. 6B illustrates quantification of human TNFα in the sample by using the microbeads through Mass Cytometry.

FIG. 7 illustrates detection of microbeads with different combinational elements through Mass Cytometry, in which the yellow arrow indicates signal of microbeads with element tag of a combination of 141Pr, 159Tb, 169Tm (without 165Ho) and coupled with CD3 primer, the red arrow indicates signal of microbeads with element tag of a combination of 141Pr, 165Ho, 169Tm (without 159Tb) and coupled with CD4 primer, the green arrow indicates signal of microbeads with element tag of a combination of 141Pr, 159Tb, 165Ho (without 169Tm) and coupled with CD19 primer.

FIG. 8A-8C illustrate quantification of nucleic acids in samples comprising H₂O, HEK293T RNA, and peripheral blood mononuclear cell (PBMC) RNA, respectively.

### DETAILED DESCRIPTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definition

As used herein and in the appended claims, the singular forms “a,” “and,” and “the” include plural referents unless the context clearly dictates otherwise. Thus, for example, reference to “a group” includes a plurality of such groups, reference to “a monomer” includes a plurality of such monomers, and reference to “the pendent moiety” includes reference to one or more pendant moieties (or to a plurality of pendant moieties) and equivalents thereof known to those skilled in the art, and so forth.

The term “about” or “approximately” herein means within an acceptable error range of the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated, the term “about” or “approximately” meaning within an acceptable error range for the particular value should be assumed.

When ranges are used herein for physical properties, such as diameter, or chemical properties, such as functional groups, all combinations and sub-combinations of ranges and specific embodiments therein are intended to be included. Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure provided herein.

The term “and/or” as used herein is a functional word to indicate that two words or expressions are to be taken together or individually. For example, A and/or B encompasses A alone, B alone, and A and B together.

The term “comprising” (and related terms such as “comprise” or “comprises” or “having” or “including”) is not intended to exclude that in other embodiments, for example, an embodiment of any composition of matter, composition, method, or process, or the like, described herein, may “consist of” or “consist essentially of” the described features.

The term “microbead” as used herein refers to a micrometer sized monodispersed particle consisting of organic polymers. It functions as a carrier for both the element tag and the target molecule to be analyzed in an element analysis. Any suitable particle with a size in the range between 10 nm-1 mm can be utilized as the microbead of the present disclosure. For example, the microbead of the present disclosure can be a particle in a size between 10 nm-1 mm, 10 nm-100 μm, 0.1 μm-10 μm, or any range in between.

The term “functional group,” “reactive group” and “reactive moiety” as used interchangeably herein, refer to specific substituents or moieties within molecules that are responsible for the characteristic chemical reactions of those molecules. The same functional group will undergo the same or similar chemical reaction(s) regardless of the size of the molecule it is a part of.

The term “chelating group” as used herein refers a molecular component comprising a functional part capable of binding to an atom or ion and can be attached to the functional group on the surface of the microbead of the present disclosure. Examples of functional part of the chelating group include but are not limited to 2-aminomethylpyridine, iminoacetic acid, iminodiacetic acid, ethylenediaminetetraacetic acid (EDTA), diethylenetriaminepentaacetic acid (DTPA), 1,4,7,10-tetraazacyclododecane-1,4,7,10-tetraacetic acid (DOTA), carbonyliminodiacetic acid, methyleneiminoacetic acid, methyleneiminodiacetic acid, ethylenethioethyleneiminoacetic acid, ethylenethioethyleneiminodiacetic acid, TMT, a terpyridinyl group, a chelating agent comprising a terpyridyl group and a carboxymethylamino group, or a salt of any of the foregoing acids. Especially preferred chelating groups are DTPA, DTPA-BMA, DPDP, TMT, DOTA and HPDO3A.

The term “element” as used herein refers to any chemical element or isotope capable of chelating with the chelating group on the surface of the microbead. Examples of the element include but are not limited to Y, Ru, Rh, Pd, Cd, In, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og, or an isotope thereof. Examples of the isotopes include but are not limited to Y-89, Rh103, Pd-102, Pd-104, Pd-105, Pd106, Pd-108, Pd-110, Cd-106, Cd-108, Cd-110, Cd-111, Cd-112, Cd-113, Cd-114, Cd-116, In-115, La-139, Ce-140, Ce-142, Pr-141, Nd-142, Nd-143, Nd-144, Nd-145, Nd-146, Nd-148, Nd-150, Sm-144, Sm-147, Sm-148, Sm-149, Sm-150, Sm-152, Sm-154, Eu-151, Eu-153, Gd-154, Gd-155, Gd-156, Gd-157, Gd-158, Gd-160, Tb-159, Dy-160, Dy-161, Dy-162, Dy-163, Dy-164, Ho-165, Er-164, Er-166, Er-167, Er-168, Er-170, Tm-169, Yb-170, Yb-171, Yb-172, Yb-173, Yb-174, Yb-176, Lu-175, Lu-176 and Bi-209. The lanthanide metals are preferred due to their low abundancy in environment. In some embodiments, the metal is a lanthanide metal and is selected from La, Ce, Pr, Nd, Pm, Sm, Eu, Gd, Tb, Dy, Ho, Er, Tm, Yb, Lu, or an isotope thereof.

The term “tag” as used herein refers to a molecule or a moiety thereof for identifying and/or quantifying a target molecule. A tag may be attached to the target molecule directly or indirectly. A tag may be attached to the target molecule through a linker, or the microbead herein. A tag may be attached to a microbead disclosed herein directly or indirectly. A tag may be attached to a microbead through a functional group disclosed herein. A tag of the present disclosure can comprise an element or a combination of elements that permits identification, recognition, and/or quantification of the target molecule to which it is attached. In some embodiments, the tag may further comprise other indicators, such as a fluorescence indicator, for example, a fluorochrome. The process of attaching a tag to the target molecule or microbead is sometimes referred to herein as “tagging” and a molecule or microbead that undergoes tagging or that contains a tag is referred to as “tagged” (e.g., “tagged microbead”). When the tag of the present disclosure is comprised by a combination of elements for use in detecting multiple target molecules in a sample simultaneously, the tag also can be referred as a “barcode.”

The term “barcode” as used herein refers to a tag comprising a combination of multiple element tags that allows identification of more than one analyte simultaneously. A predetermined combination of element tags and presence or absence of one or more particular element tags may be used to identify the presence or absence of a certain analyte. The relative abundance of each element tags bounded with the analyte may also be used to determine the absolute or relative abundance of the analyte. In some embodiments, a certain barcode is used to differentially label a subpopulation of microbeads in a population of microbeads. In some embodiments the barcode to label a subpopulation of microbeads is different to at least another barcode on another subpopulation of microbeads. In some embodiments, the barcode of each subpopulations of microbeads in a population of microbeads can be different from each other.

The term “affinity moiety” as used herein refers to any molecular component that can be coupled with the functional group on the surface of the microbead and is capable of binding to a target molecule so that to capture the target molecule from a sample. Examples of the affinity moiety include but are not limited to peptide, protein, aptamer, antibody, enzyme, avidin, streptavidin, carbohydrate, nucleic acid, deoxyribonucleic acid, oligonucleotide, polypeptide, recombinant protein, ribonucleic acid lipid, and a derivative thereof.

The term “spacer” as used herein refers to a molecular fragment which can be used to conjugate the affinity moiety or the chelating group with the functional group on the surface of the microbead. In some embodiments, the spacer can provide a suitable distance between the affinity moiety such as antibody and the microbead, so that to overcome steric hindrance between them and reduce the impact on the activity of the affinity moiety.

The term “MS” (Mass Spectrometry) is an analytical technique that ionizes chemical species and sorts the ions based on their mass-to-charge ratio. Various types of MS can be used in the present disclosure, including but are not limited to matrix-assisted laser desorption/ionization source with a time-of-flight mass analyzer (MALDI-TOF), inductively coupled plasma-mass spectrometry (ICP-MS), accelerator mass spectrometry (AMS), atomic absorption spectroscopy (AAS), thermal ionization-mass spectrometry (TIMS), quadrupole mass analyzers, ion trap analyzers, orbitrap analyzers, electrospray ionization mass spectrometry (ESI), fourier transform mass spectrometry (e.g., Fourier transform ion cyclotron resonance), tandem mass spectrometry (MS/MS), liquid chromatography mass spectrometry (LC/MS), and spark source mass spectrometry (SSMS), and Mass Cytometry.

“ICP-MS” (Inductively Coupled Plasma Mass Spectrometry) is a type of mass spectrometry which is capable of detecting element such as metals and non-metals at concentrations as low as one part in 1015 (part per quadrillion, ppq) on non-interfered low-background isotopes. This is achieved by ionizing the sample with inductively coupled plasma and then using a mass spectrometer to separate and quantify those ions.

“ICP-TOF-MS” (Time of Flight Inductively Coupled Plasma-Mass Spectrometry) also provides high precision and reliable element trace analysis detection. The detection limit of the methods ranges from the parts per million (ppm) (routine analysis for industrial production applications) to the parts per billion (ppb) or parts per trillion (ppt) levels for research and development purposes. It can also identify trace contamination and unknown chemical

“Mass Cytometry” is a next generation flow cytometry platform which utilizes elemental mass spectrometry to detect element-conjugated antibodies that are bound intracellularly or extracellularly to antigens of interest on single cells. Mass Cytometry can accurately discriminate elements such as metals and isotopes of different atomic masses without channel overlap, which ameliorates the need for complex compensation matrices and enables simultaneous analysis of a much greater number of cellular features than fluorescence flow cytometry.

The term “target molecule” as used herein refers to any of a variety of biological molecules to be analyzed. Examples of the target molecule include but are not limited to peptide, protein, aptamer, antibody, enzyme, carbohydrate, nucleic acid, deoxyribonucleic acid, oligonucleotide, polypeptide, recombinant protein, ribonucleic acid lipid, and a derivative thereof. In some embodiments, examples of the target molecule include but are not limited to RNA, DNA, oligonucleotides, modified or derivatized nucleotides, enzymes, receptors, receptor ligands (such as hormones), antibodies, antigens, and toxins, as well as cells including blood cells and tissue cells.

The term “analyte” as used herein refers to a substance of interest present in a sample. Analytes detectable in the present invention are those that can be captured by the affinity moiety of the microbead of the present disclosure. Examples of analytes includes but are not limited to cytokines, hormone, growth factors, proteins, peptides, CD antigen, viral antigen, antibody, neurotrophin, nucleic acids, oligonucleotides, and metabolites.

The term “biomarkers” as used herein refers to a molecule that is associated either quantitatively or qualitatively with a biological change. Clinically, biomarkers can be used for predicting, diagnosing a disease, or evaluating responses to a therapeutic intervention. Examples of biomarkers of the present disclosure include but are not limited to cytokines, hormone, growth factors, proteins, peptides, CD antigen, viral antigen, antibody, neurotrophin, nucleic acids, oligonucleotides, and metabolites.

The term “prevention” or “preventing,” “treatment” or “treating,” or “palliating” or “ameliorating” can be used interchangeably herein, and refer to an approach for obtaining beneficial or desired results including but not limited to a therapeutic benefit and/or a prophylactic benefit. Said therapeutic benefit refers to eradication or amelioration of the underlying disorder being treated. In some embodiments, a therapeutic benefit is achieved with the eradication or amelioration of one or more of the physiological symptoms associated with the underlying disorder such that an improvement is observed in the subject, notwithstanding that the subject may still be afflicted with the underlying disorder. In some embodiments, to achieve a prophylactic benefit, the compositions may be administered to a subject at risk of developing a disease, or to a subject reporting one or more of the physiological symptoms of a disease even though a diagnosis of this disease may not have been made.

The term “subject,” “individual” or “patient” as used herein refers to any animals that can be used in the present disclosure, including but not limited to human, primate, rodent, canine, feline, equine, ovine, porcine, and the like.

The term “in vivo” as used herein refers to an event that takes place in a subject's body.

The term “in vitro” as used herein refers to an event that takes places outside of a subject's body. In some embodiments, an in vitro assay encompasses any assay run outside of a subject assay. in vitro assays encompass cell-based assays in which cells alive or dead are employed. in vitro assays also encompass a cell-free assay in which no intact cells are employed.

### Microbeads

Simultaneous detection and quantification of multiple biomolecules in a single assay can significantly improve the efficiency of a biological analysis. However, significant challenges of the detection include capturing sufficient analyte from a small amount of sample and detecting the analyte with an approach with high sensitivity and accuracy.

In one aspect, provided herein is a microbead for detection and quantification analyte or target molecule in a sample. The structure of the microbead enables accurate, cost effective, and high throughput detection and quantification of the analyte or target molecule in the sample.

In some embodiments, the microbead of the present disclosure comprises a plurality of functional groups on surface of a microbead substrate, wherein a first subset of the plurality of the functional groups is attached to a chelating group capable of chelating with an element tag, and a second subset of the plurality of the functional groups is coupled with an affinity moiety capable of binding to a target molecule. The affinity moiety of the microbead allows capturing of a target molecule to be analyzed, whereas the chelating group of the microbead allows chelation of an element tag with the microbead for element analysis.

Any suitable polymer can be used to form the substrate of the microbead of the present disclosure. In some embodiments, the polymer that can be used to form the substrate of the microbead is a homopolymer. In some embodiments, the polymer that can be used to form the substrate of the microbead is a copolymer. Examples of the polymers that can be used to for the substrate of the microbead include but is not limited to polystyrene (PS), polystyrene-methacrylic acid (PSMAA), polystyrene-divinylbenzene (P[S/DVB]), polymethyl methacrylate (PMMA), and any combination thereof. In some embodiments, the substrate of the microbead is formed by polymerization of polystyrene (PS).

In some embodiments, the substrate of the microbead may be pre-manufactured before subjecting to functionalization. In some embodiments of the present invention, the substrate of the microbeads can be obtained from commercial suppliers.

In some embodiments, the shape of the microbead can be irregularly, or substantially spherical, cubic, rhomboid, tetragonal, dodecahedral, ovoid, cylindrical, and the like. In some embodiments, the shape of the microbead is substantially spherical.

In some embodiments, the microbead may have a size in the range between 10 nm-1 mm, or any range in between. In some embodiments, the microbead may have a size in the range between 50 nm-100 μm. In some embodiments, the microbead may have a size in the range between 50 nm-10 μm. In some embodiments, the microbead may have a size in the range between 100 nm-10 μm. In some embodiments, the microbead may have a size in the range between 300 nm-10 μm. In some embodiments, the microbead may have a size in the range between 500 nm-10 μm. In some embodiments, the microbead may have a size in the range between 100 nm-5 μm. In some embodiments, the microbead may have a size in the range between 300 nm-5 μm. In some embodiments, the microbead may have a size in the range between 500 nm-5 μm. In some embodiments, the microbead may have a size in the range between 100 nm-3 In some embodiments, the microbead may have a size in the range between 300 nm-3 μm. In some embodiments, the microbead may have a size in the range between 500 nm-3 μm. In some embodiments, the microbead may have a size in the range between 1 μm-3 μm. In some embodiments, the microbead may have a size in the range of 1 μm-10 μm. In some embodiments, the microbead may have a size in the range between 1 μm-5 μm. In some embodiments, the microbead may have a size in the range between 5 μm-10 μm.

In some embodiments, the microbead may have a diameter in the range between 10 nm-1 mm, or any range in between. In some embodiments, the microbead may have a diameter in the range between 50 nm-100 μm. In some embodiments, the microbead may have a diameter in the range between 50 nm-10 μm. In some embodiments, the microbead may have a diameter in the range between 100 nm-10 μm. In some embodiments, the microbead may have a diameter in the range between 300 nm-10 μm. In some embodiments, the microbead may have a diameter in the range between 500 nm-10 μm. In some embodiments, the microbead may have a diameter in the range between 100 nm-5 μm. In some embodiments, the microbead may have a diameter in the range between 300 nm-5 μm. In some embodiments, the microbead may have a diameter in the range between 500 nm-5 μm. In some embodiments, the microbead may have a diameter in the range between 100 nm-3 In some embodiments, the microbead may have a diameter in the range between 300 nm-3 μm. In some embodiments, the microbead may have a diameter in the range between 500 nm-3 μm. In some embodiments, the microbead may have a diameter in the range between 1 μm-3 μm. In some embodiments, the microbead may have a diameter in the range of 1 μm-10 μm. In some embodiments, the microbead may have a diameter in the range between 1 μm-5 μm. In some embodiments, the microbead may have a diameter in the range between 5 μm-10 μm.

In some embodiments, the microbeads may be substantially spherical, with a diameter between 10 nm-1 mm or any range in between. In some embodiments, the microbeads may be substantially spherical, with a diameter in the range between 50 nm-100 μm. In some embodiments, the microbeads may be substantially spherical, with a diameter in the range between 50 nm-10 In some embodiments, the microbeads may be substantially spherical, with a diameter in the range between 100 nm-10 μm. In some embodiments, the microbeads may be substantially spherical, with a diameter in the range between 300 nm-10 μm. In some embodiments, the microbeads may be substantially spherical, with a diameter in the range between 500 nm-10 μm. In some embodiments, the microbeads may be substantially spherical, with a diameter in the range between 100 nm-5 μm. In some embodiments, the microbeads may be substantially spherical, with a diameter in the range between 300 nm-5 μm. In some embodiments, the microbeads may be substantially spherical, with a diameter in the range between 500 nm-5 μm. In some embodiments, the microbeads may be substantially spherical, with a diameter in the range between 100 nm-3 In some embodiments, the microbeads may be substantially spherical, with a diameter in the range between 300 nm-3 μm. In some embodiments, the microbeads may be substantially spherical, with a diameter in the range between 500 nm-3 μm. In some embodiments, the microbeads may be substantially spherical, with a diameter in the range between 1 μm-3 μm. In some embodiments, the microbeads may be substantially spherical, with a diameter in the range between 1 μm-10 μm. In some embodiments, the microbeads may be substantially spherical, with a diameter in the range between 1 μm-5 μm. In some embodiments, the microbeads may be substantially spherical, with a diameter in the range between 5 μm-10 μm.

In some embodiments, the microbead comprises a plurality of functional groups on surface. In some embodiments, the microbead comprises 10⁴-10¹⁰ functional groups on its surface. In some embodiments, the microbead comprises 10⁵-10¹⁰ functional groups on its surface. In some embodiments, the microbead comprises 10⁶-10¹⁰ functional groups on its surface. In some embodiments, the microbead comprises 10⁷-10¹⁰ functional groups on its surface. In some embodiments, the microbead comprises 10⁸-10¹⁰ functional groups on its surface. In some embodiments, the microbead comprises 10⁹-10¹⁰ functional groups on its surface. In some embodiments, the microbead comprises 10⁴-10⁹ functional groups on its surface. In some embodiments, the microbead comprises 10⁴-10⁸ functional groups on its surface. In some embodiments, the microbead comprises 10⁴-10⁷ functional groups on its surface. In some embodiments, the microbead comprises 10⁴-10⁶ functional groups on its surface. In some embodiments, the microbead comprises 10⁵-10⁹ functional groups on its surface. In some embodiments, the microbead comprises 10⁶-10⁹ functional groups on its surface. In some embodiments, the microbead comprises 10⁷-10⁹ functional groups on its surface. In some embodiments, the microbead comprises 10⁶-10⁸ functional groups on its surface.

The functional group on the surface of the microbead can be any suitable functional group capable of attaching to the chelating group, coupling with the affinity moiety and/or a spacer. In some embodiments, examples of the functional groups include but are not limited to carboxyl, sulfhydryl, amino, hydroxyl, maleimide, azide, alkynyl, biotin and any combination thereof. In some embodiments, the functional group used on the surface of the microbead is carboxyl.

In some embodiments, the microbead of the present disclosure is substantially spherical formed by polystyrene and comprises 10⁴-10¹⁰ carboxyl groups as the functional groups on the surface of the microbead. In some embodiments, the microbead is a substantially spherical formed by polystyrene and comprises 10⁶-10⁹ carboxyl groups as the functional groups on the surface of the microbead.

In some embodiments, a first subset of the plurality of the functional groups on the surface of the microbead is attached to a chelating group. The chelating group attached to the functional group on the surface of the microbead can be any suitable chelating group capable of chelating with an element tag. In some embodiments, examples of the chelating group include but are not limited to EDTA (ethylenediaminetetraacetic acid), PDCA (2,6-pyridinedicarboxylic acid), DTPA (diethylenetriaminepentaacetic acid), DCTA (diaminocyclohexanetetraacetic acid), DOTA (1,4,7,10-Tetraazacyclododecane-1,4,7,10-tetraacetic acid), TETA (N,N′-Bis(2-aminoethyl)ethane-1,2-diamine), NOTA (1,4,7-triazacyclononane-N,N′,N″-triacetic acid), and a derivative thereof.

In some embodiments, the microbead comprises 10⁴-10¹⁰ chelating groups attached to the functional groups on the surface of the microbead. In some embodiments, the microbead comprises 10⁵-10¹⁰ chelating groups attached to the functional groups on the surface of the microbead. In some embodiments, the microbead comprises 10⁶-10¹⁰ chelating groups attached to the functional groups on the surface of the microbead. In some embodiments, the microbead comprises 10⁷-10¹⁰ chelating groups attached to the functional groups on the surface of the microbead. In some embodiments, the microbead comprises 10⁸-10¹⁰ chelating groups attached to the functional groups on the surface of the microbead. In some embodiments, the microbead comprises 10⁹-10¹⁰ chelating groups attached to the functional groups on the surface of the microbead. In some embodiments, the microbead comprises 10⁴-10⁹ chelating groups attached to the functional groups on the surface of the microbead. In some embodiments, the microbead comprises 10⁴-10⁸ chelating groups attached to the functional groups on the surface of the microbead. In some embodiments, the microbead comprises 10⁴-10⁷ chelating groups attached to the functional groups on the surface of the microbead. In some embodiments, the microbead comprises 10⁴-10⁶ chelating groups attached to the functional groups on the surface of the microbead. In some embodiments, the microbead comprises 10⁵-10⁹ chelating groups attached to the functional groups on the surface of the microbead. In some embodiments, the microbead comprises 10⁶-10⁹ chelating groups attached to the functional groups on the surface of the microbead. In some embodiments, the microbead comprises 10⁷-10⁹ chelating groups attached to the functional groups on the surface of the microbead. In some embodiments, the microbead comprises 10⁶-10⁸ chelating groups attached to the functional groups on the surface of the microbead.

In some embodiments, the chelating groups attached to the functional group on the surface of the microbead can be the same chelating groups. In some embodiments, the chelating groups attached to the functional group on the surface of the microbead can be a combination of different chelating groups, such as any combination of two, three or more of EDTA (ethylenediaminetetraacetic acid), PDCA (2,6-pyridinedicarboxylic acid), DTPA (diethylenetriaminepentaacetic acid), DCTA (diaminocyclohexanetetraacetic acid), DOTA (1,4,7,10-Tetraazacyclododecane-1,4,7,10-tetraacetic acid), TETA (N,N′-Bis(2-aminoethyl)ethane-1,2-diamine) and NOTA (1,4,7-triazacyclononane-N,N′,N″-triacetic acid).

In some embodiments, the microbead comprises 10⁴-10¹⁰ DOTA attached to the functional groups on the surface of the microbead. the microbead comprises 10⁶-10⁹ DOTA attached to the functional groups on the surface of the microbead. In some embodiments, the microbead comprises 10⁴-10¹⁰ EDTA attached to the functional groups on the surface of the microbead. the microbead comprises 10⁶-10⁹ EDTA attached to the functional groups on the surface of the microbead. In some embodiments, the microbead comprises 10⁴-10¹⁰ DTPA attached to the functional groups on the surface of the microbead. the microbead comprises 10⁶-10⁹ DTPA attached to the functional groups on the surface of the microbead. In some embodiments, the microbead comprises 10⁴-10¹⁰ combinational chelating groups on the surface of the microbead. In some embodiments, the microbead comprises 10⁶-10⁹ combinational chelating groups on the surface of the microbead.

In some embodiments, the chelating group is attached to the functional group on the surface of the microbead directly. In some embodiments, the chelating group is attached to the functional group on the surface of the microbead via a spacer. Any suitable spacer can be used to connect the chelating group to the functional group. Examples of the spacer that can be used to connect the chelating group to the functional group include but are not limited to MPBH (4-(4-N-maleimidophenyl) butyric acid hydrazide), NH₂-(PEG)ₙ-Maleimide, and a derivative thereof. In some embodiments, the spacer comprises two or more spacers. In some embodiments, the spacer comprises two or more the same spacers. In some embodiments, the spacer comprises two or more the different spacers.

In some embodiments, the chelating group of the microbead further chelates with an element tag. Any element tag suitable for an element analysis can be used for chelating with the chelating group of microbead of the present disclosure. In some embodiments, the element is a metal or an isotope thereof.

Examples of metals that can be used in the present disclosure includes but are not limited to Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og.

Examples of isotopes that can be used in the present disclosure includes but are not limited to Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, the element tag of the present disclosure comprises a combination of two or more elements or isotope thereof. In some embodiments, the element tag of the present disclosure comprises a combination of two elements or isotope thereof. In some embodiments, the element tag of the present disclosure comprises a combination of three elements or isotope thereof. In some embodiments, the element tag of the present disclosure comprises a combination of four elements or isotope thereof. In some embodiments, the element tag of the present disclosure comprises a combination of five elements or isotope thereof.

In some embodiments, the element tag of the present disclosure comprises a combination of two metals selected from Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og. In some embodiments, the element tag of the present disclosure comprises a combination of two isotopes selected from Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, the element tag of the present disclosure comprises a combination of three metals selected from Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og. In some embodiments, the element tag of the present disclosure comprises a combination of three isotopes selected from Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, the element tag of the present disclosure comprises a combination of four metals selected from Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og. In some embodiments, the element tag of the present disclosure comprises a combination of four isotopes selected from Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, a second subset of the plurality of the functional groups on the surface of the microbead is coupled with an affinity moiety. The affinity moiety can be any suitable moiety capable of binding to a target molecule. Examples of the affinity moiety include but are not limited to peptide, protein, aptamer, antibody, enzyme, avidin, streptavidin, carbohydrate, nucleic acid, deoxyribonucleic acid, oligonucleotide, polypeptide, recombinant protein, ribonucleic acid lipid, and a derivative thereof. In some embodiments, the affinity moiety is an antibody, and the antibody can be selected from monoclonal antibody, polyclonal antibody, antibody fragment, Fab fragment, Fc fragment, light chain, heavy chain, immunoglobin, and immunoglobin fragment.

In some embodiments, the affinity moieties coupled with the functional group on the surface of the microbead can be the same affinity moiety. In some embodiments, the affinity moieties coupled with the functional group on the surface of the microbead can be a combination of different affinity moieties such as a combination of two, three or more of different affinity moieties.

In some embodiments, the microbead comprises 10⁴-10¹⁰ affinity moieties coupled with the functional groups on the surface of the microbead. In some embodiments, the microbead comprises 10⁵-10¹⁰ affinity moieties coupled with the functional groups on the surface of the microbead. In some embodiments, the microbead comprises 10⁶-10¹⁰ affinity moieties coupled with the functional groups on the surface of the microbead. In some embodiments, the microbead comprises 10⁷-10¹⁰ affinity moieties coupled with the functional groups on the surface of the microbead. In some embodiments, the microbead comprises 10⁸-10¹⁰ affinity moieties coupled with the functional groups on the surface of the microbead. In some embodiments, the microbead comprises 10⁴-10⁹ affinity moieties coupled with the functional groups on the surface of the microbead. In some embodiments, the microbead comprises 10⁵-10⁹ affinity moieties coupled with the functional groups on the surface of the microbead. In some embodiments, the microbead comprises 10⁶-10⁹ affinity moieties coupled with the functional groups on the surface of the microbead. In some embodiments, the microbead comprises 10⁵-10⁸ affinity moieties coupled with the functional groups on the surface of the microbead. In some embodiments, the microbead comprises 10⁶-10⁸ affinity moieties coupled with the functional groups on the surface of the microbead. In some embodiments, the microbead comprises 10⁷-10⁸ affinity moieties coupled with the functional groups on the surface of the microbead. In some embodiments, the microbead comprises 10⁵-10⁷ affinity moieties coupled with the functional groups on the surface of the microbead. In some embodiments, the microbead comprises 10⁶-10⁷ affinity moieties coupled with the functional groups on the surface of the microbead. In some embodiments, the microbead comprises 10⁵-10⁶ affinity moieties coupled with the functional groups on the surface of the microbead.

In some embodiments, the microbead comprises 10⁴-10¹⁰ antibodies coupled with the functional groups on the surface of the microbead. the microbead comprises 10⁵-10⁶ antibodies coupled with the functional groups on the surface of the microbead. In some embodiments, the microbead comprises 10⁴-10¹⁰ antibodies coupled with the functional groups on the surface of the microbead. the microbead comprises 10⁵-10⁶ antibodies coupled with the functional groups on the surface of the microbead. In some embodiments, the microbead comprises 10⁴-10¹⁰ antibodies coupled with the functional groups on the surface of the microbead. the microbead comprises 10⁵-10⁶ antibodies coupled with the functional groups on the surface of the microbead. In some embodiments, the microbead comprises 10⁴-10¹⁰ combinational antibodies coupled with the functional groups on the surface of the microbead. In some embodiments, the microbead comprises 10⁵-10⁶ combinational antibodies coupled with the functional groups on the surface of the microbead.

In some embodiments, the affinity moiety is coupled with the functional group on the surface of the microbead directly. In some embodiments, the affinity moiety is coupled with the functional group on the surface of the microbead via a spacer. In some embodiments, the spacer provides a suitable distance between the affinity moiety such as antibody and the microbead, so that to overcome steric hindrance between them and reduce the impact on the activity of the affinity moiety.

Any suitable spacer can be used to connect the affinity moiety to the functional group. Examples of the spacer that can be used to connect the affinity moiety to the functional group include but are not limited to MPBH (4-(4-N-maleimidophenyl) butyric acid hydrazide), NH₂-(PEG)ₙ-Maleimide, and a derivative thereof.

In some embodiments, the spacer comprises two or more spacers. In some embodiments, the spacer comprises two or more the same spacers. In some embodiments, the spacer comprises two or more the different spacers.

In some embodiments, the microbead is a substantially spherical formed by polystyrene, and comprises 10⁴-10¹⁰ carboxyl groups as the functional groups on the surface of the microbead, wherein a first subset of the carboxyl groups is attached to a chelating group capable of chelating with an element tag and s second subset of the carboxyl groups is coupled with affinity moiety capable of binding to a target molecule. In some embodiments, the microbead is a substantially spherical formed by polystyrene, and comprises 10⁴-10¹⁰ carboxyl groups as the functional groups on the surface of the microbead, wherein a first subset of the carboxyl groups is attached to a chelating group chelating with an element tag comprising a combination of elements, and a second subset of the carboxyl groups is coupled with affinity moiety capable of binding to a target molecule. In some embodiments, the microbead is a substantially spherical formed by polystyrene, and comprises 10⁴-10¹⁰ carboxyl groups as the functional groups on the surface of the microbead, wherein a first subset of the carboxyl groups is attached to a chelating group chelating with an element tag comprising a combination of three elements, and a second subset of the carboxyl groups is coupled with an affinity moiety capable of binding to a target molecule. In some embodiments, the microbead is a substantially spherical formed by polystyrene, and comprises 10⁴-10¹⁰ carboxyl groups as the functional groups on the surface of the microbead, wherein a first subset of the carboxyl groups is attached to a chelating group chelating with an element tag comprising a combination of three elements, and a second subset of the carboxyl groups is coupled with an antibody capable of binding to a target molecule.

In another aspect, provided herein is a population of microbeads comprising two or more subpopulations of the microbeads, wherein at least one of the microbeads comprises a plurality of functional groups on surface of a microbead substrate, a first subset of the plurality of the functional groups is attached to a chelating group chelated with an element tag, and a second subset of the plurality of the functional groups is coupled with an affinity moiety capable of binding to a target molecule; and wherein at least one subpopulation of the microbeads comprises an element tag that is different from at least another subpopulation of the microbeads, and at least one subpopulation of the microbeads comprises an affinity moiety that is different from another subpopulation of the microbeads. Each subpopulation of microbeads can capture a corresponding target molecule, which allows the population of the microbeads capable of detecting multi-target molecule of a sample in a single assay simultaneously.

In some embodiments, the affinity moiety of one subpopulation of the microbeads binds to a target molecule that is different from the affinity moiety of another subpopulation of the microbeads. In some embodiments, the affinity moiety of one subpopulation of the microbeads binds to an epitope that is different from the affinity moiety of another subpopulation of the microbeads.

In some embodiments, the element tag of each subpopulation of the microbeads in the population of the microbeads comprises a combination of elements, and the element can be a metal or an isotope thereof. In some embodiments, the element tag of the microbead in the population of the microbeads comprises a combination of two elements. In some embodiments, the element tag of the microbead in the population of the microbeads comprises a combination of two elements selected from Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og. In some embodiments, the element tag of the microbead in the population of the microbeads comprises a combination of two elements selected from Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175. In some embodiments, the element tag of the microbead in the population of the microbeads is a combination of three elements. In some embodiments, the element tag of the microbead in the population of the microbeads comprises a combination of three elements selected from Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og. In some embodiments, the element tag of the microbead in the population of the microbeads comprises a combination of three elements selected from Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

The combination of elements can function as a barcode to enable differential identification of a subpopulation of the microbeads in the population of the microbeads by using element analysis. Using such combination of elements as the barcode of the subpopulation of the microbeads makes it possible to obtain more diverse tags by using a limited number of elements for the population of the microbeads. For example, using 10 different elements for a population of the microbeads and choosing 3 from 10 of different elements as a barcode can generate 120 different barcodes for up to 120 subpopulations in the population of the microbeads. The strategy of using the combinational elements allows multiplex analysis of a large number of analytes in a sample simultaneously in a single element analysis, which is significantly cost effective and time-saving.

In some embodiments, a combination of 2 different elements are used to function as the barcode for the subpopulation of microbeads. In some embodiments, a combination of 3 different elements are used to function as the barcode for the subpopulation of microbeads. In some embodiments, a combination of 4 different elements are used to function as the barcode for the subpopulation of microbeads. In some embodiments, a combination of 5 different elements are used to function as the barcode for the subpopulation of microbeads. In some embodiments, a combination of 6 different elements are used to function as the barcode for the subpopulation of microbeads. In some embodiments, a combination of 7 different elements are used to function as the barcode for the subpopulation of microbeads. In some embodiments, a combination of 8 different elements are used to function as the barcode for the subpopulation of microbeads. In some embodiments, a combination of 9 different elements are used to function as the barcode for the subpopulation of microbeads. In some embodiments, a combination of 10 different elements are used to function as the barcode for the subpopulation of microbeads.

In some embodiments, 2 different elements are used to generate diverse barcodes for the population of the microbeads. In some embodiments, 3 different elements are used to generate diverse barcodes for the population of the microbeads. In some embodiments, 4 different elements are used to generate diverse barcodes for the population of the microbeads. In some embodiments, 5 different elements are used to generate diverse barcodes for the population of the microbeads. In some embodiments, 6 different elements are used to generate diverse barcodes for the population of the microbeads. In some embodiments, 7 different elements are used to generate diverse barcodes for the population of the microbeads. In some embodiments, 8 different elements are used to generate diverse barcodes for the population of the microbeads. In some embodiments, 9 different elements are used to generate diverse barcodes for the population of the microbeads. In some embodiments, 10 different elements are used to generate diverse barcodes for the population of the microbeads. In some embodiments, 11 different elements are used to generate diverse barcodes for the population of the microbeads. In some embodiments, 12 different elements are used to generate diverse barcodes for the population of the microbeads. In some embodiments, 13 different elements are used to generate diverse barcodes for the population of the microbeads. In some embodiments, 14 different elements are used to generate diverse barcodes for the population of the microbeads. In some embodiments, 15 different elements are used to generate diverse barcodes for the population of the microbeads. In some embodiments, 16 different elements are used to generate diverse barcodes for the population of the microbeads. In some embodiments, 17 different elements are used to generate diverse barcodes for the population of the microbeads. In some embodiments, 18 different elements are used to generate diverse barcodes for the population of the microbeads. In some embodiments, 19 different elements are used to generate diverse barcodes for the population of the microbeads. In some embodiments, 20 different elements are used to generate diverse barcodes for the population of the microbeads. In some embodiments, 50 different elements are used to generate diverse barcodes for the population of the microbeads. In some embodiments, 100 different elements are used to generate diverse barcodes for the population of the microbeads.

In some embodiments, provided is a population of microbeads comprising a plurality of subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises an element tag with a unique combination of elements as its barcode, and comprises an affinity moiety that is different from at least another subpopulation of the microbeads.

In some embodiments, provided is a population of microbeads comprising 2 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moiety of the other subpopulation of the microbeads. In some embodiments, provided is a population of microbeads comprising 3 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other two subpopulations of the microbeads. In some embodiments, provided is a population of microbeads comprising 4 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 5 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 10 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 20 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 50 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 100 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 200 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 500 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads. In some embodiments, provided is a population of microbeads comprising 1000 or more subpopulations of the microbeads, wherein each of the microbeads comprises a plurality of functional groups on the surface, and a first subset of the plurality of the functional groups is attached to a chelating group chelated with an element tag and a second subset of the plurality of the functional groups is coupled with an affinity moiety capable of binding to a target molecule; wherein one subpopulation of the microbeads comprises a element tag with a unique combination of elements as its barcode, and comprises an affinity moiety that is different from the affinity moieties of the other subpopulations of the microbeads.

In some embodiments, the population of the microbeads has a variable coefficient of diameter (CVd) that is less than 10%. In some embodiments, the population of the microbeads has a variable coefficient of diameter (CVd) that is less than 9%. In some embodiments, the population of the microbeads has a variable coefficient of diameter (CVd) that is less than 8%. In some embodiments, the population of the microbeads has a variable coefficient of diameter (CVd) that is less than 7%. In some embodiments, the population of the microbeads has a variable coefficient of diameter (CVd) that is less than 6%. In some embodiments, the population of the microbeads has a variable coefficient of diameter (CVd) that is less than 5%. In some embodiments, the population of the microbeads has a variable coefficient of diameter (CVd) that is less than 4%. In some embodiments, the population of the microbeads has a variable coefficient of diameter (CVd) that is less than 3%. In some embodiments, the population of the microbeads has a variable coefficient of diameter (CVd) that is less than 2%. In some embodiments, the population of the microbeads has a variable coefficient of diameter (CVd) that is less than 1%.

In some embodiments, the population of the microbeads is substantially spherical, and has a variable coefficient of diameter (CVd) that is less than 10%. In some embodiments, the population of the microbeads is substantially spherical, and has a variable coefficient of diameter (CVd) that is less than 9%. In some embodiments, the population of the microbeads is substantially spherical, and has a variable coefficient of diameter (CVd) that is less than 8%. In some embodiments, the population of the microbeads is substantially spherical, and has a variable coefficient of diameter (CVd) that is less than 7%. In some embodiments, the population of the microbeads is substantially spherical, and has a variable coefficient of diameter (CVd) that is less than 6%. In some embodiments, the population of the microbeads is substantially spherical, and has a variable coefficient of diameter (CVd) that is less than 5%. In some embodiments, the population of the microbeads is substantially spherical, and has a variable coefficient of diameter (CVd) that is less than 4%. In some embodiments, the population of the microbeads is substantially spherical, and has a variable coefficient of diameter (CVd) that is less than 3%. In some embodiments, the population of the microbeads is substantially spherical, and has a variable coefficient of diameter (CVd) that is less than 2%. In some embodiments, the population of the microbeads is substantially spherical, and has a variable coefficient of diameter (CVd) that is less than 1%.

In some embodiments, the multiple affinity moieties of the population of the microbeads can be used to capture multiple target molecules in a sample simultaneously, and the element tags of the microbeads which function as barcodes of the affinity moieties can be used to identify the affinity moieties in an element analysis so that to enable analysis of the multiple target molecules simultaneously. Examples of the affinity moiety of each subpopulation of the microbeads in the population of the microbeads include but are not limited to peptide, protein, aptamer, antibody, enzyme, avidin, streptavidin, carbohydrate, nucleic acid, deoxyribonucleic acid, oligonucleotide, polypeptide, recombinant protein, ribonucleic acid lipid, and a derivative thereof. In some embodiments, the affinity moiety of each subpopulation of the microbeads in the population of the microbeads is antibody.

In some embodiments, after capturing the target molecule, the microbead or the population of the microbeads of the present disclosure can be subject to an element analysis. Any approach capable of analyzing element and isotope can be used as the element analysis of the present disclosure. In some embodiments, the element analysis is Mass Spectrometry (MS). Examples of MS that can be used for the element analysis include but are not limited to ICP-MS, ICP-TOF-MS and Mass Cytometry.

### Preparation Method

In another aspect, provided is a method for preparing the microbead of the present disclosure, comprising providing a polymeric microbead substrate; functionalizing the polymeric microbead substrate to provide a plurality of functional groups on the surface of the polymeric microbead substrate; reacting a chelating group with the polymeric microbead substrate in (b), so that the chelating group is attached to a first subset of the functional groups on surface of the polymeric microbead substrate; reacting an affinity moiety with the polymeric microbead substrate in (b), so that the affinity moiety is coupled with a second subset of the functional groups on surface of the polymeric microbead substrate.

The polymeric microbead substrate of the microbead can be formed by polymerization of any substrate. In some embodiments, the polymeric microbead substrate is provided by polymerization of styrene, styrene-methacrylic acid (SMAA), styrene-divinylbenzene (S/DVB), and methyl methacrylate (PMMA), or any combination thereof. In some embodiments, the polymeric microbead substrate is polystyrene (PS) which is provided by polymerization of styrene. In some embodiments, the polymeric microbead substrate is polystyrene-methacrylic acid (PSMAA) which is provided by polymerization of styrene-methacrylic acid (SMAA). In some embodiments, the polymeric microbead substrate is polystyrene-divinylbenzene (P[S/DVB]) which is provided by polymerization of styrene-divinylbenzene (S/DVB). In some embodiments, the polymeric microbead substrate is polymethyl methacrylate (PMMA) which is provided by polymerization of methyl methacrylate (MMA).

In some embodiments, the polymeric microbead substrate is functionalized to provide a plurality of functional groups on the surface of the polymeric microbead substrate. Examples of functional groups that can be used to functionalize the polymeric microbead substrate include but are not limited to carboxyl, sulfhydryl, amino, hydroxyl, maleimide, azide, alkynyl, biotin, and any combination thereof.

In some embodiments, 10⁴-10¹⁰ of the functional groups are provided on the surface of the polymeric microbead substrate. In some embodiments, 10⁵-10¹⁰ of the functional groups are provided on the surface of the polymeric microbead substrate. In some embodiments, 10⁶-10¹⁰ of the functional groups are provided on the surface of the polymeric microbead substrate. In some embodiments, 10⁷-10¹⁰ of the functional groups are provided on the surface of the polymeric microbead substrate. In some embodiments, 10⁸-10¹⁰ of the functional groups are provided on the surface of the polymeric microbead substrate. In some embodiments, 10⁹-10¹⁰ of the functional groups are provided on the surface of the polymeric microbead substrate. In some embodiments, 10⁴-10⁹ of the functional groups are provided on the surface of the polymeric microbead substrate. In some embodiments, 10⁴-10⁸ of the functional groups are provided on the surface of the polymeric microbead substrate. In some embodiments, 10⁴-10⁷ of the functional groups are provided on the surface of the polymeric microbead substrate. In some embodiments, 10⁴-10⁶ of the functional groups are provided on the surface of the polymeric microbead substrate. In some embodiments, 10⁵-10⁹ of the functional groups are provided on the surface of the polymeric microbead substrate. In some embodiments, 10⁶-10⁹ of the functional groups are provided on the surface of the polymeric microbead substrate. In some embodiments, 10⁷-10⁹ of the functional groups are provided on the surface of the polymeric microbead substrate. In some embodiments, 10⁶-10⁸ of the functional groups are provided on the surface of the polymeric microbead substrate.

In some embodiments, 10⁴-10¹⁰ of carboxyl groups are provided on the surface of the polymeric microbead substrate. In some embodiments, 10⁶-10⁹ of carboxyl groups are provided on the surface of the polymeric microbead substrate. In some embodiments, 10⁴-10¹⁰ of sulfhydryl groups are provided on the surface of the polymeric microbead substrate. In some embodiments, 10⁶-10⁹ of sulfhydryl groups are provided on the surface of the polymeric microbead substrate. In some embodiments, 10⁴-10¹⁰ of carboxyl groups are provided on the surface of the polymeric microbead substrate. In some embodiments, 10⁶-10⁹ of carboxyl groups are provided on the surface of the polymeric microbead substrate. In some embodiments, 10⁴-10¹⁰ of azide groups are provided on the surface of the polymeric microbead substrate. In some embodiments, 10⁶-10⁹ of azide groups are provided on the surface of the polymeric microbead substrate. In some embodiments, 10⁴-10¹⁰ of biotin groups are provided on the surface of the polymeric microbead substrate. In some embodiments, 10⁶-10⁹ of biotin groups are provided on the surface of the polymeric microbead substrate.

In some embodiments, the method for preparing the microbead of the present disclosure comprises reacting a chelating group with the polymeric microbead substrate so that the chelating group is attached to a first subset of the functional groups on surface of the polymeric microbead substrate. Any suitable chelating group capable of chelating an element tag can be used to attach to the functional groups on the surface of the microbead. Examples of the chelating groups include but are not limited to EDTA, DTPA, DCTA, DOTA, TETA, NOTA, and a derivative thereof.

In some embodiments, the chelating group is attached to the functional group via an amine of the chelating group. In some embodiments, the amine is a primary amine. In some embodiments, the chelating group is attached to the functional group through carbodiimide crosslinking. In some embodiments, the carbodiimide crosslinking comprises activating the functional group by a carbodiimide compound before attaching the chelating group to the functional group. Examples of the carbodiimide compound include but are not limited to EDC (1-Ethyl-3-[3-dimethylaminopropyl] carbodiimide hydrochloride) and DCC (dicyclohexyl carbodiimide). In some embodiments, the carbodiimide crosslinking further comprises forming an NETS-ester intermediate before attaching the chelating group to the functional group. In some embodiments, the NETS-ester intermediate is formed by NHS (N-hydroxy-succinimide). In some embodiments, the NETS-ester intermediate is formed by Sulfo-NHS (N-hydroxy-sulfo-succinimide).

In some embodiments, the chelating group is attached to the functional group via a spacer. Examples of spacers that can be used to connect the chelating group to the functional group include but are not limited to MPBH (4-(4-N-maleimidophenyl) butyric acid hydrazide) and NH₂-(PEG)ₙ-Maleimide. In some embodiments, the spacer comprises two or more spacers. In some embodiments, the spacer comprises two or more the same spacers. In some embodiments, the spacer comprises two or more the different spacers.

In some embodiments, the method further comprises chelating an element tag to the chelating group. In some embodiments, the element tag comprises an element. In some embodiments, the element is a metal or an isotope thereof.

Examples of metals that can be used in the present disclosure includes but are not limited to Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og.

Examples of isotopes that can be used in the present disclosure includes but are not limited to Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, the element tag comprises a combination of elements. In some embodiments, the element tag comprises a combination of two or more elements. In some embodiments, the element tag comprises a combination of three or more elements. In some embodiments, the elements of the combination are chelated to the chelating group in a fix ratio. In some embodiments, the elements of the combination are chelated to the chelating group in a random ratio. In some embodiments, the elements of the combination are chelated to the chelating group in equal ratio. In some embodiments, the elements of the combination are chelated to the chelating group in non-equal ratio.

In some embodiments, the method comprises reacting an affinity moiety with the polymeric microbead substrate so that the affinity moiety is coupled with a second subset of the functional groups on surface of the polymeric microbead substrate. Any suitable affinity moiety capable of binding to a target molecule can be used to couple with the functional group on the surface of the microbead. Examples of the affinity moiety include but are not limited to peptide, protein, aptamer, antibody, enzyme, avidin, streptavidin, carbohydrate, nucleic acid, deoxyribonucleic acid, oligonucleotide, polypeptide, recombinant protein, ribonucleic acid lipid, and a derivative thereof. In some embodiments, the antibody includes but is not limited to monoclonal antibody, polyclonal antibody, antibody fragment, Fab fragment, Fc fragment, light chain, heavy chain, immunoglobin, and immunoglobin fragment.

In some embodiments, the affinity moiety is coupled with the functional group via an amine of the affinity moiety. In some embodiments, the amine is a primary amine. In some embodiments, the affinity moiety is coupled with the functional group through carbodiimide crosslinking. In some embodiments, the carbodiimide crosslinking comprises activating the functional group by a carbodiimide compound before attaching the chelating group to the functional group. Examples of the carbodiimide compound include but are not limited to EDC (1-Ethyl-3-[3-dimethylaminopropyl] carbodiimide hydrochloride) and DCC (dicyclohexyl carbodiimide). In some embodiments, the carbodiimide crosslinking further comprises forming an NETS-ester intermediate before coupling the affinity moiety with the functional group. In some embodiments, the NETS-ester intermediate is formed by NHS (N-hydroxy-succinimide). In some embodiments, the NETS-ester intermediate is formed by Sulfo-NHS (N-hydroxy-sulfo-succinimide).

In some embodiments, the affinity moiety is coupled with the functional group via a spacer. Examples of spacers that can be used to connect the affinity moiety to the functional group include but are not limited to MPBH (4-(4-N-maleimidophenyl) butyric acid hydrazide) and NH₂-(PEG)ₙ-Maleimide. In some embodiments, the spacer comprises two or more spacers. In some embodiments, the spacer comprises two or more the same spacers. In some embodiments, the spacer comprises two or more the different spacers.

In some embodiments, the affinity moiety is coupled with the functional group before attaching the chelating group to the functional group. In some embodiments, the affinity moiety is coupled with the functional group after attaching the chelating group to the functional group. In some embodiments, the affinity moiety is coupled with the functional group before chelating the element tag to the chelating group. In some embodiments, the affinity moiety is coupled with the functional group after chelating the element tag to the chelating group.

In some embodiments, provided is a method for preparing a population of microbeads comprising two or more subpopulation of the microbeads. In some embodiments, the method for preparing a population of microbeads comprises preparing each subpopulation of the microbeads comprising a unique element barcode and affinity moiety first, and then mixing all subpopulations of the microbeads together to form the population of the microbeads.

### Method of Use

In another aspect, provided is a method for detecting an analyte in a sample, comprising contacting the sample with a microbead, wherein the microbead comprises a plurality of functional groups on surface of a microbead substrate, and a first subset of the plurality of the functional groups is attached to a chelating group chelated with an element tag and a second subset of the plurality of the functional groups is coupled with an affinity moiety capable of binding to the analyte; differentiating the microbead bound to the analyte from the microbead not bound to the analyte; determining the analyte in the sample. In some embodiments, the analyte is determined based on the element tag of the microbeads.

In some embodiments, the differentiating comprises incubating the microbead with an immobilizing reagent, wherein the immobilizing reagent specifically binds to the analyte and immobilizes the microbead bound to the analyte. Any suitable immobilizing reagent capable of specifically binding to the analyte can be used to differentiate the microbead bound to the analyte. In some embodiments, the immobilizing reagent is a surface comprising an affinity component conjugated on the surface capable of specifically binding to the analyte. Examples of the surface include but are not limited to the surface of a substrate, a matrix, a particle, a bead, a plate, a chip, which comprises an affinity component conjugated on the surface capable of specifically binding to the analyte. In some embodiments, the affinity component binds to a molecular surface of the analyte that is different from the affinity moiety of the microbead of the present disclosure. In some embodiments, the affinity component binds to an epitope of the analyte that is different from the affinity moiety of the microbead of the present disclosure.

In some embodiments, the method further comprises isolating the microbeads bound to the analyte before determining the analyte by an element analysis. In some embodiments, the microbeads bound to the analyte is immobilized by the surface as described above and the microbeads not bound to the analyte can be washed away to isolate the microbeads bound to the analyte from those unbound. In some embodiments, the method further comprises releasing the microbeads immobilized by the surface before determining the analyte by an element analysis.

In some embodiments, the differentiating comprises incubating the microbead with a detecting reagent, wherein the detecting reagent specifically binds to the analyte and allows for detection of the microbead bound to the analyte. In some embodiments, the method further comprises isolating the microbead from the sample. In some embodiments, the microbead is isolated from the sample by centrifuge.

Examples of the detecting reagent include but are not limited to peptide, protein, aptamer, antibody, enzyme, carbohydrate, nucleic acid, deoxyribonucleic acid, oligonucleotide, polypeptide, recombinant protein, ribonucleic acid lipid, and a derivative thereof. In some embodiments, the detecting reagent is an antibody. In some embodiments, the detecting antibody specifically binds to a molecular surface of the analyte that is different from the affinity moiety of the microbead of the present disclosure. In some embodiments, the detecting antibody specifically binds to an epitope of the analyte that is different from the affinity moiety of the microbead of the present disclosure.

In some embodiments, the detecting reagent is labeled with a detecting tag which allows detection of the microbead bound to the analyte. The detecting tag can be any suitable tag which allows differentiation of the microbead. In some embodiments, the detecting tag is a detecting element tag. In some embodiments, the detecting element tag is a metal or an isotope thereof.

Examples of the metals that can be used in the detecting element tag include but are not limited to Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og.

Examples of the isotopes that can be used in the detecting element tag include but are not limited to Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, the method for detecting an analyte in a sample comprises contacting the sample with a microbead of the present disclosure, wherein a plurality of the functional groups of the microbead are attached to a chelating group chelated with an element tag and a plurality of the functional groups of the microbead are coupled with an affinity moiety capable of binding to the analyte; differentiating the microbead bound to the analyte from the microbead not bound to the analyte with a detecting reagent capable of specifically binding to the analyte, wherein the detecting reagent comprises a detecting element tag which allow detection of the microbead bound to the analyte by an element analysis.

In some embodiments, the element analysis is Mass Spectrometry (MS). Examples of MS that can be used for the element analysis include but are not limited to ICP-MS, ICP-TOF-MS and Mass Cytometry.

In some embodiments, the method for detecting the analyte comprises determining the analyte in the sample. In some embodiments, the determining the analyte comprises determining an identity of the analyte in the sample. In some embodiments, the determining the identity of the analyte is performed by analyzing the identity of the element tag. In some embodiments, the determining the analyte is performed by analyzing the microbead bound to the analyte by the detecting element tag, and the identity of the analyte in the sample by the element tag of the microbead.

In some embodiments, the determining the analyte comprises quantifying the amount of the analyte in the sample. In some embodiments, the quantifying the amount of the analyte is performed by analyzing the detecting element tag. In some embodiments, the quantifying the amount of the analyte is performed by the relative amount of the detecting element tag. In some embodiments, the quantifying the amount of the analyte is performed by the absolute amount of the detecting element tag.

In some embodiments, the method can be used to analyze a sample obtained from a subject. In some embodiments, the sample is bodily fluid of the subject. Examples of the bodily fluid include but are not limited to whole blood, plasma, serum, urine, effusions, ascitic fluid, saliva, cerebrospinal fluid, cervical secretions, vaginal secretions, endometrial secretions, amniotic fluid, gastrointestinal secretions, bronchial secretions including sputum, breast fluid and secretions.

In some embodiments, the method described above can be used to analyze one or more biomarkers in the sample. In some embodiments, the biomarkers can be used for predicting or diagnosing a disease. Examples of the biomarkers include but are not limited to cytokines, chemokines, hormone, growth factors, proteins, peptides, CD antigen, viral antigen, antibody, neurotrophin, nucleic acids, oligonucleotides, and metabolites.

In some embodiments, the method of the present disclosure can be used to analyze nucleic acid in a sample in liquid or on solid substrate. In some embodiments, the method of the present disclosure can be used to analyze nucleic acids in cell samples, RNA samples, DNA samples or any combination thereof. Additional enzymes may be applied for analyzing different samples. For example, Taq enzyme, other DNA polymerase and its variants can be used for analyzing nucleic acids in cell or cell-free samples.

In some embodiments, examples of the biomarkers include but are not limited to interleukins such as IL-1a, IL-1b, IL-2, IL-3, IL-4, IL-5, IL-6 to IL-36; interferons such as IFNα, IFNβ, IFNγ; growth factors such as M-CSF, G-CSF, GM-CSF, EGF, VEGF, FGF, GDNF, HGF, IGF, TGF; and receptor ligands such as OX40L, CD40L, FASL, CD27L, CD30L, 4-1BBL, TRAIL.

In some embodiments, examples of cytokines that can be analyzed by the method of the present disclosure include but are not limited to IFN-alpha, IFN-gamma, TNF-alpha, TNF-beta, IL-2, IL-3, IL-4, IL-5, IL-6, IL-7, IL-8, IL-9, IL-10, IL-11, IL-12, IL-13, IL-15, IL-1 alpha, IL-1 beta, CNTF, LIF, OSM, EPO, G-CSF, PRL, GM-CSF, M-CSF, SCF, aFGF, bFGF, INT-2, KGF, EGF, Betacelluli, SCDGF, Amphiregulin, HB-EGF, 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin, Otoraplin, Resistin, Serum Amyloid A, TPO, Trefoil Factor, TSLP, Tumor Necrosis Factor, Uteroglobin, Visfatin, and the like.

In some embodiments, examples of chemokines that can be analyzed by the method of the present disclosure include but are not limited to CCL14, CCL19, CCL20, CCL21, CCL25, CCL27, CXCL12, CXCL13, CXCL-8, CCL2, CCL3, CCL4, CCL5, CCL11, CXCL10, CCR1, CCR2, CCR3, CCR4, CCR5, CCR6, CCR7, CCR8, CCR9, CCR10, CCR10A, CCR10B, CCR11, ECRF-3, EBI-1 (EBV induced gene-1), US28, MCP-4, MIP-4, 1-309, TECK, GCP-2, Mig, lymphotactin, MCP-2, MCP-3, eotaxin-1, MCIF, TARC, MDC, MPIF-2 (eotaxin-2), SDF-1 and fractalkine (neurotactin), and the like.

In some embodiments, examples of soluble CD antigens that can be analyzed by the method of the present disclosure include but are not limited to sCD4, sCD8, sCD23, sCD25, sCD27, sCD30, sCD52, and the like.

In some embodiments, examples of growth factors that can be analyzed by the method of the present disclosure include but are not limited to Activin, CSF, CTGF, Epigen, Erythropoietin Fibroblast Growth Factor, Galectin, HDGF, Hepatocyte Growth Factor, IGFBP Insulin-Like Growth Factor, Insulin, Keratinocyte Growth Factor, Leptin, Macrophage Migration Inhibitory Factor, Melanoma Inhibitory Activity Myostatin, Noggin, NOV, Omentin, Oncostatin-M, Osteopontin, Osteoprotegerin, PDGF, Periostin, Placental Growth Factor, Placental Lactogen, Prolactin PRL RANK Ligand, Retinol Binding Protein, Stem Cell Factor, Transforming Growth Factor, VEGF, EGF, FGF, NGF, PDGF, VEGF, IGF, GMCSF, GCSF, TGF, Erythropieitn, TPO, BMP, HGF, GDF, Neurotrophins, MSF, SGF, GDF, and the like.

In some embodiments, examples of hormones that can be analyzed by the method of the present disclosure include but are not limited to Endothelin, Exendin, FSH, GHRP, GLP, Glucagon, HCG, Inhibin A, LHRH, ACTH, Alarelin, Antide, Atosiban, Buserelin, Cetrorelix, DDAVP, Deslorelin, Elcatonin, Ganirelix, GHRL Protein, Goserelin, Hexarelin, Histrelin, Lanreotide, Leuprorelin Human, Lypressin, Melanotan-I, Melanotan-II, NAF, PMSG, Pramlintide, Secretin, Sincalide, Somatostatin, Terlipressin, Thymopentin, Procalcitonin, PTH, Stanniocalcin, Thymosin, Triptorelin Acetate, Thyrostimulin, TSH, Vasopressin and the like.

In another aspect, provided is a method for performing a multiplex analysis of two or more analytes in a sample comprising: (a) contacting a population of microbeads with the sample, wherein each of the microbeads comprises a plurality of functional groups on surface of the microbead substrate; a first subset of the plurality of the functional groups is attached to a chelating group chelated with an element tag; a second subset of the plurality of the functional groups is coupled with an affinity moiety capable of binding to an analyte of the two or more analytes, wherein at least one subpopulation of the microbeads comprises the element tag that is different from at least another subpopulation of the microbeads, and at least one subpopulation of the microbeads comprises the affinity moiety that is different from another subpopulation of the microbeads, (b) incubating the population of the microbeads with two or more detecting reagents, wherein each of the detecting reagents allows for detection of a microbead bound to an analyte; (c) simultaneously analyzing the two or more analytes in the sample.

In some embodiments, the element tag of the each microbead of the population of microbeads is a combination of elements. In some embodiments, the is a combination of two or more elements. In some embodiments, the is a combination of three or more elements. In some embodiments, the is a combination of four or more elements. In some embodiments, the is a combination of five or more elements. Using such combination of elements makes it possible to obtain more diverse tags by using a limited number of elements. For example, choosing 3 from 10 of different elements can generate 120 different combination of elements, i.e. 120 different element tags. The strategy of using the combinational elements allows multiplex analysis of a large number of analytes in a sample simultaneously in a single element analysis, which is significantly cost effective and time-saving.

Examples of the detecting reagent include but are not limited to peptide, protein, aptamer, antibody, enzyme, carbohydrate, nucleic acid, deoxyribonucleic acid, oligonucleotide, polypeptide, recombinant protein, ribonucleic acid lipid, and a derivative thereof. In some embodiments, the detecting reagent is an antibody. In some embodiments, the detecting antibody specifically binds to a molecular surface of the analyte that is different from the affinity moiety of the microbead of the present disclosure. In some embodiments, the detecting antibody specifically binds to an epitope of the analyte that is different from the affinity moiety of the microbead of the present disclosure.

In some embodiments, the detecting reagent is labeled with a detecting element tag. The detecting element tag allows differentiation of the microbead bound to the analyte from the microbead not bound to the analyte. In some embodiments, the detecting element tag is a metal or an isotope thereof.

Examples of the metals that can be used in the detecting element tag include but are not limited to Y, Ru, Rh, Pd, Cd, In, Ba, La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, and Og.

Examples of the isotopes that can be used in the detecting element tag include but are not limited to Y-89, Rh-103, Pd-102, Pd-104, Pd-105, Pd-106, Pd-108, Pd-110, Cd-106, Cd-108, Cd-110, Cd-111, Cd-112, Cd-113, Cd-114, Cd-116, In-115, La-139, Ce-140, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, and Lu-175.

In some embodiments, element analysis for the multiplex analysis can be Mass Spectrometry (MS). Examples of MS include but are not limited to ICP-MS, ICP-TOF-MS and Mass Cytometry.

In some embodiments, the multiplex analysis comprises determining the identities of the two or more analytes in the sample. In some embodiments, the identities of the two or more analytes are determined by analyzing the element tag of the microbead. In some embodiments, the identities of the two or more analytes are determined by analyzing the element tag of the microbead and the detecting element tag. In some embodiments, the multiplex analysis comprises quantifying the amounts of the two or more analytes in the sample. In some embodiments, the quantification is determined by analyzing the detecting element tag.

In some embodiments, the method for performing a multiplex analysis further comprises isolating the microbeads from the sample. In some embodiments, the isolation is performed by centrifuge.

In some embodiments, the multiplex analysis can be used to analyze a sample obtained from a subject. In some embodiments, the sample is bodily fluid of the subject. Examples of the bodily fluid include but are not limited to whole blood, plasma, serum, urine, effusions, ascitic fluid, saliva, cerebrospinal fluid, cervical secretions, vaginal secretions, endometrial secretions, amniotic fluid, gastrointestinal secretions, bronchial secretions including sputum, breast fluid and secretions.

In some embodiments, the multiplex analysis described above can be used to analyze multiple biomarkers simultaneously in the sample. In some embodiments, the biomarkers can be used for predicting, diagnosing a disease, or evaluating responses to a therapeutic intervention.

Examples of the biomarkers include but are not limited to cytokines, hormone, growth factors, proteins, peptides, CD antigen, viral antigen, antibody, neurotrophin, nucleic acids, oligonucleotides, and metabolites. In some embodiments, examples of the biomarkers include but are not limited to interleukins such as IL-1a, IL-1b, IL-2, IL-3, IL-4, IL-5, IL-6 to IL-36; interferons such as IFNα, IFNβ, IFNγ; growth factors such as M-CSF, G-CSF, GM-CSF, EGF, VEGF, FGF, GDNF, HGF, IGF, TGF; and receptor ligands such as OX40L, CD40L, FASL, CD27L, CD30L, 4-1BBL, TRAIL.

Examples of cytokines that can be analyzed by the multiplex analysis of the present disclosure include but are not limited to IFN-alpha, IFN-gamma, TNF-alpha, TNF-beta, IL-2, IL-3, IL-4, IL-5, IL-6, IL-7, IL-8, IL-9, IL-10, IL-11, IL-12, IL-13, IL-15, IL-1 alpha, IL-1 beta, CNTF, LIF, OSM, EPO, G-CSF, PRL, GM-CSF, M-CSF, SCF, aFGF, bFGF, INT-2, KGF, EGF, Betacelluli, SCDGF, Amphiregulin, HB-EGF, 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin, Otoraplin, Resistin, Serum Amyloid A, TPO, Trefoil Factor, TSLP, Tumor Necrosis Factor, Uteroglobin, Visfatin, and the like.

Examples of chemokines that can be analyzed by the multiplex analysis of the present disclosure include but are not limited to CCL14, CCL19, CCL20, CCL21, CCL25, CCL27, CXCL12, CXCL13, CXCL-8, CCL2, CCL3, CCL4, CCL5, CCL11, CXCL10, CCR1, CCR2, CCR3, CCR4, CCR5, CCR6, CCR7, CCR8, CCR9, CCR10, CCR10A, CCR10B, CCR11, ECRF-3, EBI-1 (EBV induced gene-1), US28, MCP-4, MIP-4, 1-309, TECK, GCP-2, Mig, lymphotactin, MCP-2, MCP-3, eotaxin-1, MCIF, TARC, MDC, MPIF-2 (eotaxin-2), SDF-1 and fractalkine (neurotactin), and the like.

Examples of growth factors that can be analyzed by the multiplex analysis of the present disclosure include but are not limited to Activin, CSF, CTGF, Epigen, Erythropoietin Fibroblast Growth Factor, Galectin, HDGF, Hepatocyte Growth Factor, IGFBP Insulin-Like Growth Factor, Insulin, Keratinocyte Growth Factor, Leptin, Macrophage Migration Inhibitory Factor, Melanoma Inhibitory Activity Myostatin, Noggin, NOV, Omentin, Oncostatin-M, Osteopontin, Osteoprotegerin, PDGF, Periostin, Placental Growth Factor, Placental Lactogen, Prolactin PRL RANK Ligand, Retinol Binding Protein, Stem Cell Factor, Transforming Growth Factor, VEGF, EGF, FGF, NGF, PDGF, VEGF, IGF, GMCSF, GCSF, TGF, Erythropieitn, TPO, BMP, HGF, GDF, Neurotrophins, MSF, SGF, GDF, and the like.

Examples of hormones that can be analyzed by multiplex analysis of the present disclosure include but are not limited to Endothelin, Exendin, FSH, GHRP, GLP, Glucagon, HCG, Inhibin A, LHRH, ACTH, Alarelin, Antide, Atosiban, Buserelin, Cetrorelix, DDAVP, Deslorelin, Elcatonin, Ganirelix, GHRL Protein, Goserelin, Hexarelin, Histrelin, Lanreotide, Leuprorelin Human, Lypressin, Melanotan-I, Melanotan-II, NAF, PMSG, Pramlintide, Secretin, Sincalide, Somatostatin, Terlipressin, Thymopentin, Procalcitonin, PTH, Stanniocalcin, Thymosin, Triptorelin Acetate, Thyrostimulin, TSH, Vasopressin and the like.

### Kit

In another aspect, provided is a kit comprising an instruction for performing a multiplex analysis of two or more analytes by using the kit, a population of microbeads comprising two or more subpopulations of the microbeads, wherein at least one of the microbeads comprises a plurality of functional groups on surface of a microbead substrate, a first subset of the plurality of the functional groups are attached to a chelating group chelated with an element tag, and a second subset of the plurality of the functional groups is coupled with an affinity moiety capable of binding to a target molecule, and wherein at least one subpopulation of the microbeads comprises an element tag that is different from at least another subpopulation of the microbeads, and at least one subpopulation of the microbeads comprises an affinity moiety that is different from another subpopulation of the microbeads, and two or more detecting reagents, wherein each of the detecting reagents comprises a detecting element tag and specifically binds to an analyte of the two or more analytes.

In some embodiments, the kit comprises two or more subpopulations of the microbeads for capturing two or more analytes in a sample. In some embodiments, the kit comprises three or more subpopulations of the microbeads for capturing three or more analytes in a sample. In some embodiments, the kit comprises four or more subpopulations of the microbeads for capturing four or more analytes in a sample. In some embodiments, the kit comprises five or more subpopulations of the microbeads for capturing five or more analytes in a sample. In some embodiments, the kit comprises ten or more subpopulations of the microbeads for capturing ten or more analytes in a sample. In some embodiments, the kit comprises twenty or more subpopulations of the microbeads for capturing twenty or more analytes in a sample. In some embodiments, the kit comprises fifty or more subpopulations of the microbeads for capturing fifty or more analytes in a sample. In some embodiments, the kit comprises one hundred or more subpopulations of the microbeads for capturing one hundred analytes in a sample. In some embodiments, the kit comprises two hundred or more subpopulations of the microbeads for capturing two hundred or more analytes in a sample.

In some embodiments, the kit comprises two or more detecting reagents. In some embodiments, the kit comprises three or more detecting reagents. In some embodiments, the kit comprises four or more detecting reagents. In some embodiments, the kit comprises five or more detecting reagents. In some embodiments, the kit comprises ten or more detecting reagents. In some embodiments, the kit comprises twenty or more detecting reagents. In some embodiments, the kit comprises fifty or more detecting reagents. In some embodiments, the kit comprises one hundred or more detecting reagents. In some embodiments, the kit comprises two hundred or more detecting reagents.

In some embodiments, the kit of the present disclosure further comprises pharmaceutical excipients for performing the analysis. Examples of the excipients include but are not limited to stabilizer, buffer, preservative, tonicity agent, and antioxidant.

In some embodiments, examples of the buffer that can be included in the kit of the present disclosure include but are not limited to borate buffer, citrate buffer, tartrate buffer, phosphate buffer, acetate buffer and a Tris-HCl buffer (comprising tris(hydroxymethyl) aminomethane and HCl).

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1: Preparation of the Microbead

The microbead substrates were prepared by polymerization of polystyrene (PS) to yield spherical microbead substrate with an average diameter of about 1.9 μm. The surface of the microbead substrates was further functionalized with carboxyl groups, approximately 10⁹ carboxyl groups per microbead. Then the carboxyl groups were subject to further reaction.

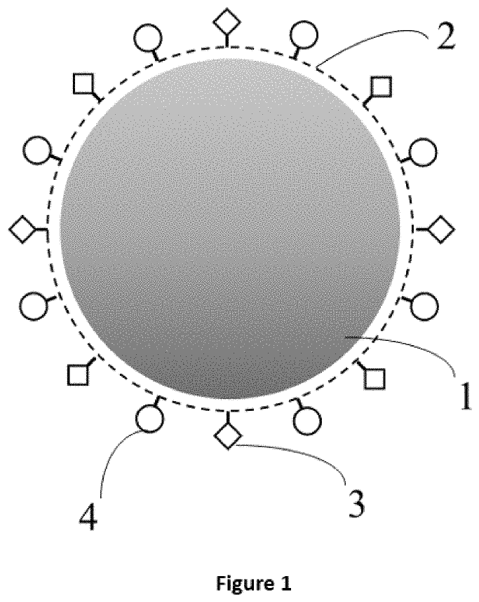

FIG. 1 illustrates the process for preparing the microbead. In the process, the microbead substrate [1] was prepared by polymerization of polystyrene (PS), and the surface of the microbead substrate was functionalized with a plurality of carboxyl groups [2], then the chelating group [3] capable of chelating with an element tag was attached to a first subset of the carboxyl groups and the affinity moiety[4] capable of binding a target molecule was coupled with a second subset of the carboxyl groups.

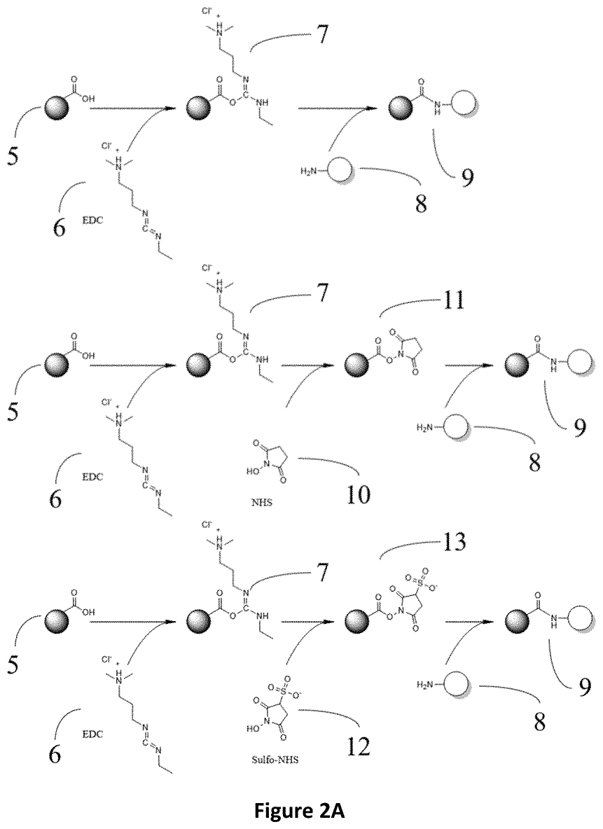

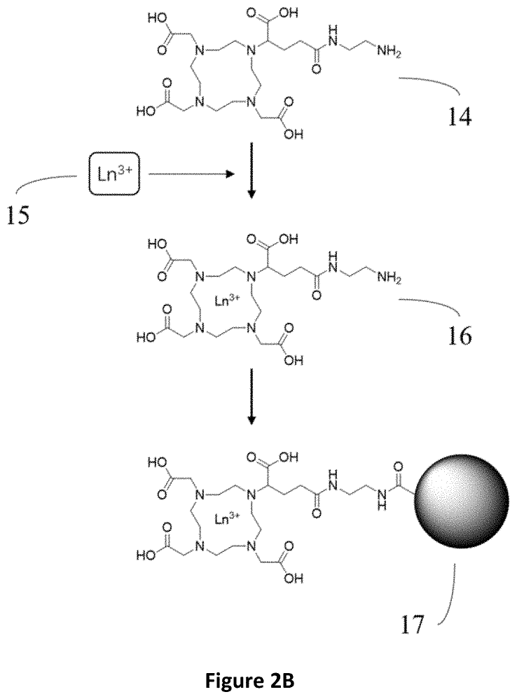

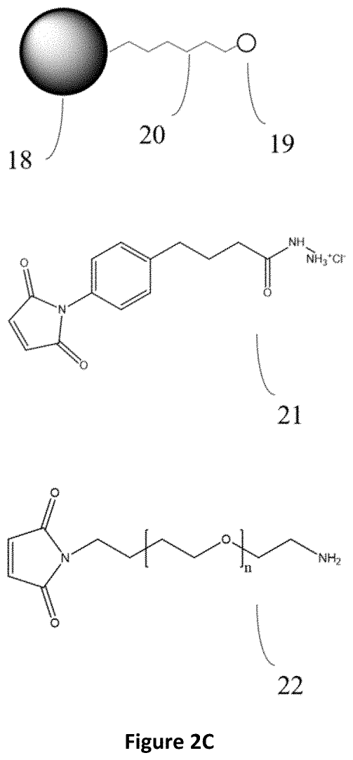

FIG. 2A illustrates the exemplary processes for coupling the affinity moiety with the functional groups on the surface of the microbead. In these processes, carbodiimide compound EDC (1-Ethyl-3-[3-dimethylaminopropyl] carbodiimide hydrochloride) [6] was employed to activate the carboxyl groups on the surface of the microbead [5] to form a reactive intermediate [7] capable of reacting with the primary amine of an affinity moiety. This reactive intermediate [7] was further reacted with the affinity moiety [8] so that to obtain the microbead coupled with the affinity moiety [9].

In an alternative process, after forming the reactive intermediate [7] by using EDC, NHS (N-hydroxy-succinimide) [10] were added to form an NETS-ester intermediate [11] capable of reacting with the primary amine of the affinity moiety. The NETS-ester intermediate [11] was further reacted with the affinity moiety [8] so that to obtain the microbead coupled with the affinity moiety [9].

In another process, after forming the reactive intermediate [7] by using EDC, Sulfo-NHS (N-hydroxy-sulfo-succinimide) [12] were added to form a sulfo-NHS-ester intermediate [13] capable of reacting with the primary amine of the affinity moiety. The sulfo-NHS-ester intermediate [13] was further reacted with the affinity moiety [8] so that to obtain the microbead coupled with the affinity moiety [9].

FIG. 2B illustrates exemplary processes for attaching the chelating groups to the functional groups on the surface of the microbead. A bifunctional chelator [14] was employ to as the chelating group. Said bifunctional chelator comprises an amino group for coupling with the carboxy group on the surface of the microbead, and a cation chelation group capable of binding to an element tag, such as a Ln3+ cation or a combination of elements [15]. In this process, the bifunctional chelator reacted with the Ln3+ first to form a chelator binding with metal cation [16], and then the chelator binding with metal cation were reacted with the carboxyl group on the surface of the microbead through the amino group of the chelator to obtain the microbead with the chelating group [17]. Alternatively, the bifunctional chelator [14] can react with the carboxyl group on the surface of the microbead first, and then subject to reaction with the element tag, such as a Ln3+ cation or a combination of elements [15] to obtain the microbead with the chelating group [17].

FIG. 2C shows an exemplary spacer that can be used to conjugate the affinity moiety to the functional group on the surface of the microbead. The spacer [20] can form a covalent linkage between the microbeads [18] and affinity moiety [19]. This spacer could provide a suitable distance between the affinity moiety such as biomolecules and the microbead, so that to overcome steric hindrance between them and reduce the impact on the activity of the affinity moiety. The spacer can be a compound MPBH (4-(4-N-maleimidophenyl) butyric acid hydrazide) [21], or functional oligomer NH₂-(PEG)ₙ-Maleimide [22]. Both of [21] and [22] have an amino group conjugating with carboxyl of the microbeads, and maleimide group conjugating with sulfhydryl group of biomacromolecules (antibodies).

### Example 2: Analysis of the Microbead with a Combinational Element Tag by Using ICP-TOF-MS

Carboxyl modified microbeads labeled with an element tag comprising a combination of elements (141Pr, 159Tb, 165Ho) were prepared by the process as described in Example 1, without coupling with the affinity moiety. Features of the prepared microbeads are as shown in Table 1.

TABLE 1
Features of the microbeads

| Element tags | Pr141, Tb159, Ho165 | |
|---|---|---|
| Ave. Diameter | 1.9 | μm |
| CVd (%) | 3.4% | |
| Carboxyl | 140 | μmol/g |

containing

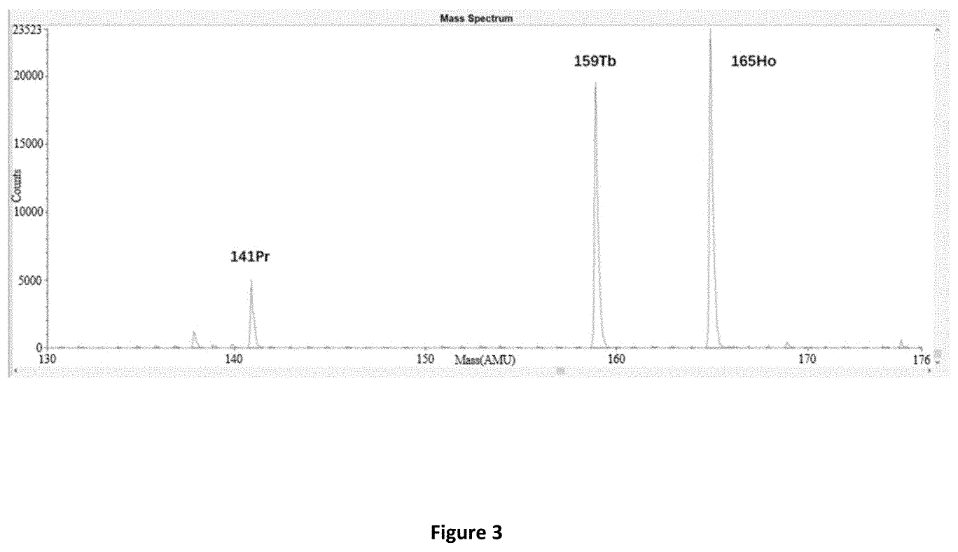

The above prepared microbeads were resuspended in distilled water at a concentration of 1×10⁶ beads/mL, and then analyzed by ICP-TOF-MS. The mass spectrum result is as shown in FIG. 3 with a Range of Mass (AMU) from 130 to 176 at 1 second accumulation. The peaks of each elements were further quantified by the integral of each peak at 1 second accumulation, as shown in Table 2.

TABLE 2
Quantification of the elements

| Element | Count/s | Ratio |
|---|---|---|
| 141Pr | 4987 | 1 |
| 159Tb | 19578 | 3.9 |
| 165Ho | 23523 | 4.7 |

### Example 3: Analysis of Microbead with a Combinational Element Tag and Mouse IgG by Mass Cytometry

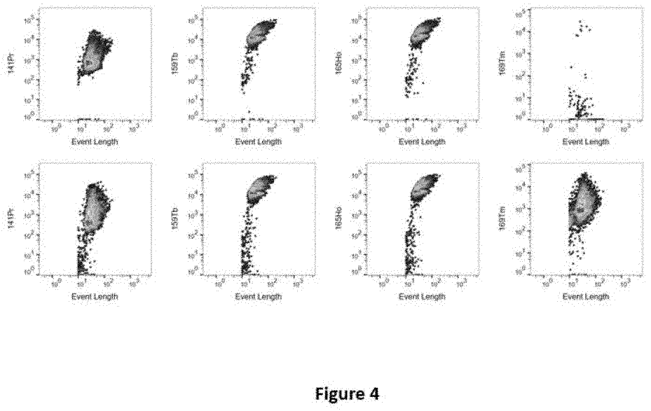

Carboxyl modified microbeads labeled with an element tag comprising a combination of elements (141Pr, 159Tb, 165Ho) were prepared by the process as described in Example 1, and the microbeads were further coupled with mouse IgG. Then the microbeads were incubated with biotin labeled anti-mouse IgG antibody and 169Tm labeled Streptavidin to obtain the microbeads binding to the anti-mouse IgG with a detecting element tag 169Tm. The microbeads were then subject to Mass Cytometry (lower panel of FIG. 4), together with the microbead prepared in Example 2 with the combinational element tag only without coupling with mouse IgG as control (upper panel of FIG. 4).

FIG. 4 shows the result of the of the Mass Cytometry. As can be seen from FIG. 4, the microbeads coupled with mouse IgG and incubated with the anti-mouse IgG with the detecting element tag 169Tm can be differentiated from the microbead without mouse IgG through the detecting element tag 169Tm, which confirms the function of the detecting element tag in differentiating microbead bound to the analyte from the microbead not bound to the analyte.

Each of the elements including 141Pr, 159Tb, 165Ho and 169Tm was further quantified based on the median and mean intensity of each element in FIG. 4. The result of the quantification is as shows in Table 3A and Table 3B.

TABLE 3A
Quantification of the elements on the microbeads
with combinational element tag only.

| | Median | Mean |
|---|---|---|
| 141Pr | 689 | 1154 |
| 159Tb | 11279 | 13668 |
| 165Ho | 13976 | 16478 |
| 169Tm | −0.00445 | 8.62 |

TABLE 3B
Quantification of the elements on the microbeads
with combinational element tag and mouse IgG.

| | Median | Mean |
|---|---|---|
| Pr141 | 610 | 1532 |
| Tb159 | 12184 | 17000 |
| Ho165 | 14843 | 19841 |
| Tm169 | 1012 | 1776 |

### Example 4: Coupling Efficiency of the Affinity Moiety with the Microbeads

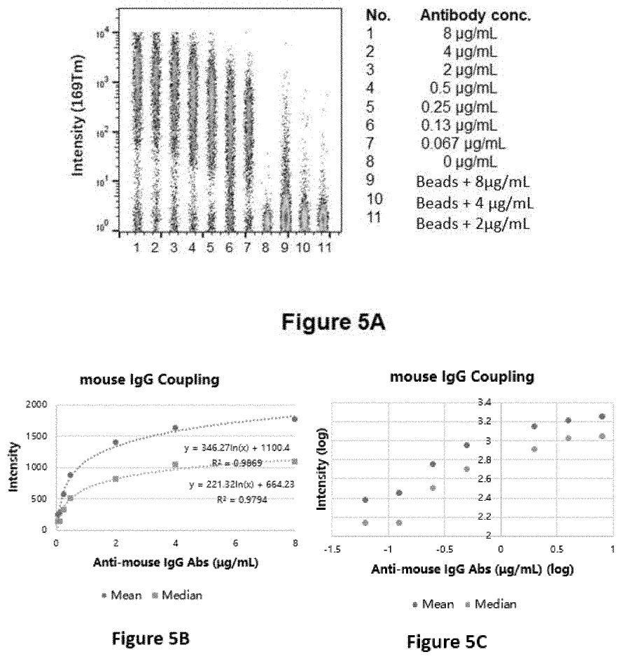

Carboxyl modified microbeads labeled with an element tag comprising a combination of elements (141Pr, 159Tb, 165Ho) and coupled with mouse IgG were prepared as described above. Then the microbeads were incubated with a gradient concentration of 169Tm labeled anti-mouse IgG antibody (2, 4 and 8 μg/mL), respectively. After the incubation, the microbeads were further resuspended in distilled water at 1×10⁶ beads/mL, and subject to Mass Cytometry. 169 Tm labeled anti-mouse IgG at different concentrations (0, 0.067, 0.13, 0.25, 0.5, 2, 4, and 8 μg/mL) were used as positive controls. Microbeads without mouse IgG were used as negative control (data not shown). The result is as shown in FIG. 5A.

The detecting element tag 169Tm in each group was further quantified in both a linear coordinate and a logarithmic coordinate, as shown in FIG. 5B and FIG. 5C, respectively.

### Example 5: Analysis of Human TNFα by Using the Method of the Present Disclosure

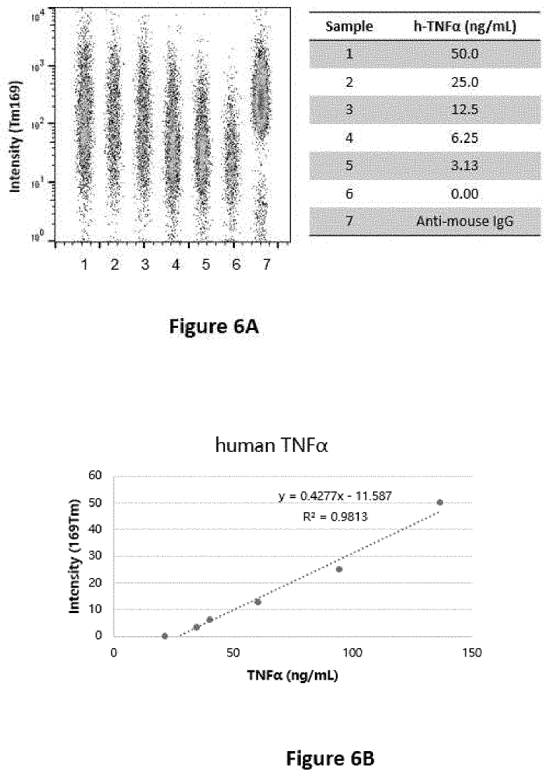

Carboxyl modified microbeads labeled with an element tag comprising a combination of elements (141Pr, 159Tb, 165Ho) and coupled with anti-human TNFα antibody were prepared as described in Example 1. Then the microbeads were incubated with samples comprising a gradient concentration of human TNFα (0, 3.13, 6.25, 12.5, 25.0, 50.0 ng/mL). Biotin labeled anti-human TNFα antibody and 169Tm labeled streptavidin (as a detecting element tag) were further added to the suspension of microbeads. Then the microbeads were subject to Mass Cytometry analysis. FIG. 6A illustrates analysis of human TNFα by using the Mass Cytometry. As can be seen from FIG. 6A, the method of the present application can detect human TNFα in the sample to a concentration as low as 3.13 ng/mL, which proves the high sensitivity of the method of the present application.

The detecting element tag 169Tm was further quantified based on the median intensity of in FIG. 6A, as shown in FIG. 6B.

### Example 6: Analysis of Nucleic Acid by Using the Method of the Present Disclosure

Element tags comprising a combination of elements selected from 141Pr, 159Tb, 165Ho, and 169Tm were coupled with carboxyl modified microbeads as described in Example 1. Then the microbeads labelled with 141Pr, 159Tb, 169Tm were further coupled with NH₂-TAG-CD3 primer-F for targeting CD3, in which TAG is a specific nucleic acid sequence consisting of T base, A base and G base. The microbeads labelled with 141Pr, 165Ho, 169Tm were further coupled with NH2-TAG-CD4 primer-F for targeting CD4. The microbeads labelled with 141Pr, 159Tb, 165Ho were further coupled with NH2-TAG-CD19 primer-F for targeting CD19.

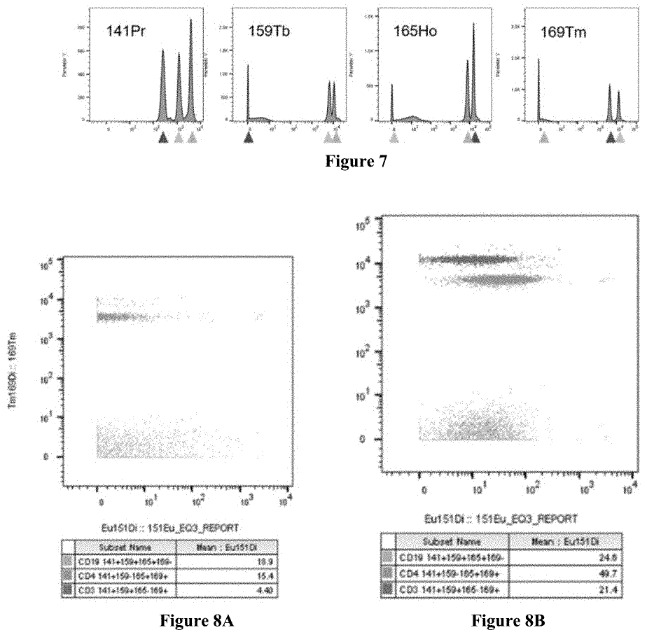

The element signal of 141Pr, 159Tb, 165Ho, 169Tm in above three varieties of microbeads were quantified by mass cytometer, as shown in FIG. 7. In FIG. 7, the yellow arrow indicates signal of microbeads with element tag of a combination of 141Pr, 159Tb, 169Tm (without 165Ho) and coupled with CD3 primer, the red arrow indicates signal of microbeads with element tag of a combination of 141Pr, 165Ho, 169Tm (without 159Tb) and coupled with CD4 primer, and the green arrow indicates signal of microbeads with element tag of a combination of 141Pr, 159Tb, 165Ho (without 169Tm) and coupled with CD19 primer. From FIG. 7, it can be seen that microbeads with different combination of elements and coupled with primers targeting different genes can be distinguished through Mass Cytometry analysis.

Then a mixture of above three varieties of microbeads comprising different combinational elements and primers were incubated with samples including H₂O (control), HEK293T RNA and peripheral blood mononuclear cell (PBMC) RNA, respectively, together with PCR mix A containing biotin-primer-R, dNTP, buffer, enzyme, RNase/DNase free water for PCR reaction. Alternatively, PCR mix B containing primer-R, biotin-dCTP, dATP, dTTP, dGTP, buffer, enzyme, RNase/DNase free water can also be used for the reaction.

After PCR, microbeads in the sample were purified by 0.25 μm column and then resuspended and incubated in buffer containing SA-Eu151 detecting element tag for a few more minutes, followed by analysis through mass cytometer.

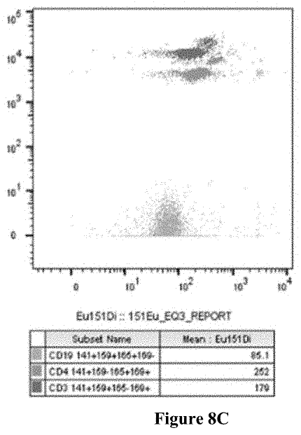

FIGS. 8A-8C show Eu151 median values detected from samples comprising H₂O, HEK293T RNA, and peripheral blood mononuclear cell (PBMC) RNA through Mass Cytometry analysis. The Eu151 median values were further quantified, as shown in Table 4.

TABLE 4
Quantification of the Eu151 on microbeads
with combinational of element tags

| | CD3- | CD4- | CD19- |
|---|---|---|---|
| | Eu151 | Eu151 | Eu151 |
| | mean | mean | mean |
| H₂O | 4.4 | 15.4 | 18.9 |
| HEK293T RNA | 21.4 | 49.7 | 24.6 |
| PBMC RNA | 179 | 252 | 85.1 |

FIGS. 8A-8C and Table 4 indicate that CD3, CD4 and CD19 were all highly expressed in PBMC, however, the expression levels of CD3, CD4 and CD19 in HEK 293 cells were all relatively low. These findings were consistent with the known characteristics of these cells, indicating the accuracy and sensitivity of the method of the present application in quantifying nucleic acid samples.

1-64. (canceled)

65. A population of microbeads comprising two or more subpopulations of the microbeads, wherein at least one of the microbeads comprises a plurality of functional groups on surface of a microbead substrate, a first subset of the plurality of the functional groups is attached to a chelating group chelated with an element tag, and a second subset of the plurality of the functional groups is coupled with an affinity moiety capable of binding to a target molecule; and wherein at least one subpopulation of the microbeads comprises an element tag that is different from at least another subpopulation of the microbeads, and at least one subpopulation of the microbeads comprises an affinity moiety that is different from another subpopulation of the microbeads.

66. The population of microbeads of claim 65, wherein the element tag is a combination of elements.

67. The population of microbeads of claim 66, wherein the combination of elements is a combination of two or more elements.

68. (canceled)

69. The population of microbeads of claim 65, wherein the element is a metal or an isotope thereof.

70. The population of microbeads of claim 65, wherein the element tag of each subpopulation of the microbeads differentiates the subpopulation of element tag from the other subpopulations.

71. The population of microbeads of claim 70, wherein the element analysis is MS.

72. The population of microbeads of claim 71, wherein the MS is selected from the group consisting of ICP-MS, ICP-TOF-MS and Mass Cytometry.

73-97. (canceled)

98. A method of performing a multiplex analysis of two or more analytes in a sample, comprising:
(a) contacting a population of microbeads with the sample, wherein at least one of the microbeads comprises a plurality of functional groups on surface of the microbead substrate; a first subset of the plurality of the functional groups is attached to a chelating group chelated with an element tag; a second subset of the plurality of the functional groups is coupled with an affinity moiety capable of binding to an analyte of the two or more analytes, wherein at least one subpopulation of the microbeads comprises the element tag that is different from at least another subpopulation of the microbeads, and at least one subpopulation of the microbeads comprises the affinity moiety that is different from another subpopulation of the microbeads,
(b) incubating the population of the microbeads with two or more detecting reagents, wherein each of the detecting reagents allows for detection of a microbead bound to an analyte;
(c) simultaneously analyzing the two or more analytes in the sample.

99. The method of claim 98, wherein the element tag is a combination of elements.

100. The method of claim 99, wherein the combination of elements is a combination of two or more elements.

101. (canceled)

102. The method of claim 98, wherein the method further comprises isolating the microbead from the sample.

103. The method of claim 98, wherein the detecting reagent is labeled with a detecting element tag.

104. The method of claim 98, wherein the analyzing the two or more analytes comprises determining the identities of the two or more analytes in the sample.

105. The method of claim 104, wherein the determining the identities of the two or more analytes is performed by analyzing the identity of the element tag.

106. (canceled)

107. (canceled)

108. The method of claim 98, wherein the analyzing the two or more analytes is performed by an element analysis.

109. The method of claim 108, wherein the element analysis is MS.

110. The method of claim 109, wherein the MS is selected from the group consisting of ICP-MS, ICP-TOF-MS and Mass Cytometry.

111-113. (canceled)

114. The method of claim 98, wherein each of the two or more analytes is a biomarker for predicting or diagnosing a disease.

115. The method of claim 114, wherein the biomarker is selected from the group consisting of cytokines, chemokines, growth factors, proteins, peptides, nucleic acids, oligonucleotides, and metabolites.

116. A kit comprising:
(1) an instruction for performing a multiplex analysis of two or more analytes by using the kit,
(2) a population of microbeads comprising two or more subpopulations of the microbeads, wherein at least one of the microbeads comprises a plurality of functional groups on surface of a microbead substrate, a first subset of the plurality of the functional groups are attached to a chelating group chelated with an element tag, and a second subset of the plurality of the functional groups is coupled with an affinity moiety capable of binding to a target molecule, and wherein at least one subpopulation of the microbeads comprises an element tag that is different from at least another subpopulation of the microbeads, and at least one subpopulation of the microbeads comprises an affinity moiety that is different from another subpopulation of the microbeads, and
(3) two or more detecting reagents, wherein each of the detecting reagents comprises a detecting element tag and specifically binds to an analyte of the two or more analytes.
